(19) United States
(12) Patent Application Publication
(10) Pub. No.: US 2005/0032246 A1
(43) Pub. Date: Feb. 10, 2005

(54) Method of immobilizing membrane-associated molecules

(75) Inventors: John D. Brennan, Dundas (CA); Michael A. Brook, Ancaster (CA); Travis Besanger, Dundas (CA)

Correspondence Address: BERESKIN AND PARR, SCOTIA PLAZA, 40 KING STREET WEST-SUITE 4000 BOX 401, TORONTO, ON, M5H 3Y2, CA

(73) Assignee: McMaster University, Hamilton (CA)

(21) Appl. No.: 10/815,727

(22) Filed: Apr. 2, 2004

Related U.S. Application Data
(63) Continuation-in-part of application No. 10/712,015, filed on Nov. 14, 2003.
(60) Provisional application No. 60/426,018, filed on Nov. 14, 2002.

(51) Int. Cl. A61L 2/00; G01N 33/543; C12P 21/06

(52) U.S. Cl. 436/518; 427/2.11

(57) ABSTRACT

The present invention relates to methods of immobilizing membrane-associated molecules within a sol-gel matrix. The membrane-associated molecule is embedded in the bilayer of a liposome. The molecule-liposome assembly remains functionally intact when it is immobilized within a protein and membrane-compatible sol-gel derived from polyol silane precursors or sodium silicate. The activity and stability of the entrapped membrane-associated molecule was significantly improved in macroporous silica.

### FIELD OF THE INVENTION

The present invention relates to methods for the immobilization of membrane-associated molecules, including membrane-associated biomolecules, to composites prepared by such methods and to the use of these composites, in particular for high-throughput drug screening, multianalyte biosensing or bioaffinity chromatography.

### BACKGROUND TO THE INVENTION

Immobilization of natural cellular receptors, which are mainly membrane associated proteins, is receiving substantial attention in the areas of research, clinical and environmental analysis, and in drug development.^{1,2,3,4,5,6,7,8,9,10,11} This is a result of increasing demand for robust and portable devices for medical, environmental and bioprocess monitoring. Just as the immobilization of biomolecules such as polynucleotides in the microarray platform has revolutionized the area of genomics, the immobilization of proteins will provide the same advantage to proteomics.^{12,13,14,15} Furthermore, immobilization of proteins provides additional advantages in the area of small molecule drug-screening using both microarray¹⁶ and chromatographic platforms.¹⁷ Thusfar, protein immobilization has focused mainly on soluble proteins, which are usually more robust than their membrane-bound counterparts. The major problems limiting the development of new sensors and high-throughput screening technologies that utilize these cellular receptors arise due to the inherently low stability of such receptors and the difficulties associated with transducing receptor-ligand binding events into measurable signals. However, membrane-bound receptors are particularly attractive targets for the development of new diagnostic devices and for discovery of new therapeutic treatments and drugs. Therefore, robust and facile immobilization techniques are needed to accommodate the sensitive supramolecular assemblies of proteins, and other membrane-associated molecules, within lipid bilayers.

A number of strategies have been reported for the immobilization of bilayer lipid membranes (BLMs) and membrane-bound receptors, including supporting of BLMs on the pores of filter paper,¹⁸ covalent attachment of monolayer or bilayer lipid membranes to surfaces,^{1,4,5,19} tethering of phospholipid liposomes to a surface by deposition,²⁰ covalent attachment¹² or via avidin-biotin linkages,²² and entrapment of BLMs into polymer multilayers to provide a semihydrated internal surface to allow incorporation of bulkier membrane receptors and proteins.⁵ For example, Vogel et al. have provided a number of reports demonstrating the immobilization and stabilization of various membrane receptors, including the G-protein coupled receptor (GPCR) neurokinin-1 and the membrane ion-channels OmpF and the nicotinic acetylcholine receptor (nAChR), on gold surfaces that were modified with a thiolipid layer.^{23,24,25,} Wainer et al. have also shown that membrane-bound receptors can be immobilized onto commercially available artificial immobilized membrane (AIM) beads, and are suitable for drug screening using an affinity chromatography format.²⁶ Additional reports have also begun to surface describing the potential of immobilized receptors for screening in the microarray format.²⁷ In this latter report, several GPCRs and their associated lipids were printed directly onto γ-aminoproplysilane derivatized slides and were able to bind to fluorescently labeled ligands.

Previous immobilization methods have been observed to reduce the natural dynamic motions of the bilayers, and lead to unstable immobilized structures.^{28,29,30,31,32,33,34,35} Problems can also arise due to the coupling of the lipid bilayer to the solid support, which can produce an unstable structure with a lifetime that is too short for functional purposes. Furthermore, the structure of intrinsic membrane-proteins relies on hydrophobic interactions internal to the lipid bilayer, as well as hydrophilic interactions on either side of the lipid membrane.⁵ Very often with conventional supported BLMs, what would be considered the hydrophilic interior surface for the membrane protein is replaced by the solid substrate.^{5,36} This situation results in destabilization of the membrane protein with a concomitant loss in activity, or in the worst-case scenario complete loss of activity due to full denaturation of the protein. These issues have been partially addressed by covalent attachment of a lipid monolayer to a solid substrate, which alleviates membrane dissociation; however, this method does not address the second issue mentioned, and furthermore decreases the natural dynamic behavior of the bilayer.^{4,5}

An emerging method for the immobilization of biological species is their entrapment within inorganic matrixes formed by the sol-gel processing method.^{37,38} This method involves formation of a colloidal sol solution owing to hydrolysis of a precursor such as tetraethyl orthosilicate (TEOS). A buffered solution containing the biomolecule of interest is then added to the sol to initiate rapid polycondensation of the silane. Following polycondensation a hydrated gel is produced that immobilizes the biological element without the need for a covalent tether.

Entrapment of soluble proteins in sol-gel derived silicate has proven to be an advantageous method for maintaining protein dynamics and activity over periods of months or more. However, the sol-gel method has found much less use for the immobilization of membrane-bound proteins. Indeed, only a few reports exist describing the immobilization of liposomes^{39,40,41,42,43} or whole cells^{44,45,46,47,48,49} into inorganic silica matrixes formed by the sol-gel method, and only a single membrane-associated protein, the photo-active receptor bacteriorhodopsin (bR), has been successfully entrapped in sol-gel derived silica.^{50,51,52,53} However, even these reports describe the entrapment of bR that was associated with only its intrinsic lipids, rather than bR that was reconstituted into a phospholipid bilayer membrane. Furthermore, the activity of entrapped bR was assessed by monitoring the decay from a photoactivated conformational intermediate referred to as the M-state, and did not directly measure ligand binding or ion channeling by entrapped bR. Limitations in the ability to monitor the ion channel activity of membrane proteins entrapped in sol-gel derived silicate may have arisen due to the selection of sol-gel precursor. During the hydrolysis of the silane precursors tetraethylorthosilicate (TEOS) of tetramethyl orthosilicate (TMOS) ethanol or methanol are produced. These byproducts will readily dissolve or destabilize existing bilayer structures.⁴³ Without a stable liposome, ion flux or membrane potential cannot be developed and therefore cannot be measured.

For commercial applications, there remains a need for a method of entrapping membrane-associated molecules as liposome assemblies that maintain the stability of both the membrane associated molecule and the bilayer lipid membrane.

### SUMMARY OF THE INVENTION

A new method for the immobilization of membrane-associated proteins or ionophore-liposome assemblies has been developed. This method is based on the immobilization of a reconstituted molecule-liposome assembly within a sol-gel-derived matrix that is prepared from protein- and membrane-compatible precursors, such as diglycerylsilane (DGS) and sodium silicate. Specifically, the ion channel proteins, gramicidin A (gA) and nicotinic acetylcholine receptor (nAChR), as well as the Ca(II) ionophore ionomycin, embedded within the membranes of 1,2-dioleoyl-sn-glycero-3-phosphocholine (DOPC) liposomes or more complex liposomes, were immobilized into DGS- or sodium silicate-derived sol-gel materials and it was shown that, upon immobilization, the gramicidin, nAChR or ionomycin remained embedded in the phospholipid membrane and their ligand-binding ability and/or transmembrane ion flux activity was retained. In addition, gramicidin remained sensitive to the concentration of ions across the membrane and selective to passage of monovalent cations through the peptide channel, ionomycin retained the ability to transport Ca(II) across the membrane, while nAChR retained its ability to transport Ca(II) across the membrane in a ligand-gated fashion based on its interaction with agonists. Furthermore, following immobilization of gramicidin, the ability of divalent cations to block ion flux through the channel was also retained, while nAChR retained the ability to be inhibited by known antagonists, which block the ion channel, indicating that modulation of membrane-channel proteins is possible following entrapment in sol-gel derived silica. Furthermore, nAChR and the dopamine D2 receptor (D2R, an example of a GPCR) were trapped in a wide variety of sol-gel derived materials (both mesoporous and meso/macroporous) and for the first time the measurement of ligand binding constants for an entrapped membrane-bound protein was determined using conventional radioligand binding assays. A significant improvement in receptor activity (ca. 70% relative to solution) and a significant decrease in non-specific binding was obtained when nAChR or D2R were entrapped into macroporous silicates formed via spinodal decomposition of added PEO (PEO, 10 kDa). Moreover, it was evident that samples retained significant activity upon storage and could be reused.

Accordingly, the present invention relates to a method of immobilizing membrane-associated molecules in silica matrixes comprising combining a liposome-assembly comprising the membrane-associated molecule, with a protein- and membrane-compatible sol-gel precursor under conditions which allow a gel to form. In an embodiment of the invention, the membrane-associated molecule and sol-gel precursor are combined with one or more additives which causes spinodal decomposition (phase transition) before gelation, to provide macroporous silica matrixes.

The present invention further relates to protein- and membrane-compatible sol-gels with a liposome-membrane associated molecule assembly immobilized therein.

Further included within the scope of the present invention are methods for the detection of modulators of membrane-associated molecules comprising:
(a) exposing a liposome assembly comprising the membrane-associated molecule, said assembly being immobilized in a protein- and membrane-compatible sol-gel, to one or more test substances; and
(b) detecting a change in one or more characteristics of the membrane-associated molecule.
   In embodiments of the invention the protein- and membrane-compatible sol-gel is prepared using a method as described herein. In further embodiments of the invention, a change in the one or more characteristics of the membrane-associated molecule in the presence of the one or more test substances compared to a control indicates that the one or more test substances may bind and/or modulate the membrane-associated molecule.

The methods of entrapment and for detecting modulators of membrane-associated molecules of the present invention provide a general method for analyzing these molecules and their inhibitors, agonists and/or antagonists. The ability to immobilize membrane-associated molecules will allow development of bioaffinity chromatography or microarray technologies that will be useful for high throughput screening of potential inhibitors or effectors.

Additionally, a novel procedure amenable to the sol-gel method of entrapment has been developed to monitor ion flux through an entrapped membrane-associated molecule. In this method, the fluorescence indicator used to detect the development of a potential (due to ion flux) across the lipid membrane was located on the inside of the liposome assembly only. Literature methods describe the use of fluorescent indicators in the both the internal and external solution. Problems caused by interactions of the indicator molecule with the anionic surface of the silica can arise when the indicator molecules are in the external solution. Such problems are avoided when the indicator molecules are located within the interior of the liposome assembly since, in this location, these molecules are not able to interact with the silica surfaces. Accordingly, the present invention further relates to an improved method for preparing a sol gel immobilized liposome assembly comprising a membrane associated molecule, wherein the membrane-associated molecule is an ion-channel molecule, comprising:
(a) obtaining a solution of the liposome assembly having an indicator molecule located on the interior of the assembly;
(b) removing the indicator molecule from solution external to the liposome assembly; and
(c) combining the liposome assembly solution with a silica precursor solution under conditions which allow a gel to form.

The present invention also relates to an improved method for the detection of membrane potentials in a sol-gel immobilized liposome assembly comprising a membrane-associated molecule, wherein the membrane-associated molecule is an ion-channel molecule, comprising:
(a) obtaining a solution of the liposome assembly having an indicator molecule located on the interior of the assembly;
(b) removing the indicator molecule from solution external to the liposome assembly;
(c) combining the liposome assembly solution with a silica precursor solution under conditions which allow a gel to form;
(d) contacting the gel with the ion and optionally a test substance; and
(e) detecting a change in the indicator molecule upon transmembrane ion flux.

The present invention also includes kits, biosensors, microarrays, chromatographic and bioaffinity columns comprising the silica matrixes that entrap a liposome-protein assembly prepared as described herein.

Yet another aspect of the present invention provides a method of conducting a target discovery business comprising:
(a) providing one or more assay systems for identifying test substances by their ability to effect one or more membrane-associated molecules based systems, said assay systems using a method of the invention;
(b) (optionally) conducting therapeutic profiling of the test substances identified in step (a) for efficacy and toxicity in animals; and
(c) licensing, to a third party, the rights for further drug development and/or sales or test substances identified in step (a), or analogs thereof.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows tryptophan emission spectra of gramicidin A before and after reconstitution into phospholipid vesicles comprised of DOPC, both in solution and after entrapment into DGS derived silica.

FIG. 2 is a schematic of the response of safranine O to the development of a membrane potential caused by an influx of potassium ions under various conditions. (a) Conventional method with safranine O located on the exterior of the liposome and (b) the “inverted” method, employing safranine O on the interior of the liposome.

FIG. 3 shows graphs indicating the change in steady-state fluorescence intensity (panel A) and anisotropy (panel B) of safranine O as membrane potential is developed across DOPC liposomes containing 0.39 mol % gramicidin A. Response follows influx of potassium ions after addition of liposomes to a solution of KI.

FIG. 4 contains graphs showing the potential-induced decrease in fluorescence intensity as a result of the influx of potassium ions into unilamellar DOPC liposomes containing various levels of gramicidin A (a) in solution and (b) following entrapment in DGS derived silicate. Units are normalized as the ratio of intensity observed at time zero.

FIG. 5 contains graphs showing the effect of different potassium iodide concentrations on the potential induced fluorescence response of safranine O for liposomes containing 0.39 mol % gramicidin A (a) in solution and (b) after entrapment in DGS derived silica. Inset for (a) depicts typical time trials for the various potassium iodide concentrations. Data are normalized as the ratios of final and initial fluorescence intensities.

FIG. 6 is a graph showing the inhibition of potassium ion flux through DOPC liposomes containing 0.39 mol % gramicidin A as a result of adding various concentrations of calcium ions to liposomes in the presence of 3 M KI.

FIG. 7 is a graph showing the aging of samples containing DOPC liposomes with 0.39 mol % gramicidin A after entrapment in DGS derived silica in the presence of 25% glycerol in distilled deionized water (●), in distilled deionized water (▪) or without any external buffer or solution (π). Data are normalized as a percentage of response observed on day 1.

FIG. 8 shows the response obtained upon addition of ³H-epibetadine to Torpedo californica nAChR entrapped in sodium silicate derived silica (Panel A) and the response obtained for blank liposomes entrapped in sodium silicate derived materials (Panel B).

FIG. 9 shows the specific binding of ³H-epibetadine to human nAChR when entrapped in DGS derived materials relative to the binding obtained in the absence of entrapped nAChR.

FIG. 10 shows the results of a competitive binding assay wherein varying concentrations of a non-radioactive antagonist (d-tubocurarine, Panel A) or agonist (nicotine, Panel B) is introduced along with 2.5 nM ³H-epibetadine to IMR-32 nAChR entrapped in DGS derived materials. The IC₅₀ and K₁ values are in good agreement with those obtained from solution based experiments.

FIG. 11 shows the concept of the fluo-3 based assay for measuring the Ca(II) ion flux across nAChR doped liposomes. In the absence of an agonist the channel remains closed and no ion flux is observed. Upon binding of an agonist the nAChR ion channel opens and Ca(II) can pass into the membrane, resulting in a large increase in emission intensity from intraliposomal Fluo-3.

FIG. 12 shows the changes in emission intensity of intraliposomal fluo-3 with time (Panel A) and the normalized concentration-dependent decrease in fluo-3 emission intensity (Panel B) due to blockage of the passage of Ca(II) ions upon addition of the antagonist d-tubocurarine to n-AChR doped liposomes entrapped in DGS derived glasses that were previously incubated with an excess of the agonist nicotine. The decease in emission intensity correlates to a decrease in ion flux owing to closing of the nAChR channel upon binding the antagonist.

FIG. 13 shows the changes in emission intensity of intraliposomal fluo-3 with time (Panel A) and the normalized concentration-dependent decrease in fluo-3 emission intensity (Panel B) due to enhanced passage of Ca(II) ions upon addition of the agonist cytisine to n-AChR doped liposomes entrapped in DGS derived glasses that were previously incubated with an excess of the antagonist d-tubocurarine. The increase in emission intensity correlates to an increase in ion flux owing to opening of the nAChR channel upon binding the agonist.

FIG. 14 is a graph showing the fluorescence intensity response of the calcium selective indicator dye Fluo-3 to the influx of calcium into DOPC liposomes in buffered solution following the addition of a calcium selective ionophore ionomycin to the membrane.

FIG. 15 is a graph showing the response of fluo-3 to the addition of calcium ions for DOPC liposomes both with and without ionomycin present within the membrane following entrapment in sodium silicate derived silica.

FIG. 16 are pictures showing a microarray of sol-gel entrapped liposomes containing ionomycin, and shows that our entrapment and signalling methods are amenable to the microarray format. Panel A shows the array before addition of calcium, panel B show the array after addition of calcium. Column 1 and 5 contain Fluo-3 loaded DOPC liposomes with ionomycin; column 2 contains only buffered sodium silicate derived silica; column 3 contains entrapped fluorescein-dextran; column 4 contains fluo-3 loaded DOPC liposomes without ionomycin.

FIG. 17 shows nAChR receptor activity as a function of ³H-epibatidine binding in various silica compositions. Light and dark grey bars indicate the difference of unbound ³H-epibatidine with and without the presence of 1.0 mM nicotine. Black bars represent the receptor specific counts, which is the difference between the receptor and liposome unbound counts. Stock receptor used: (60 nM), samples were prepared and measured as described in the experimental section.

FIG. 18 shows competitive binding of (−)-nicotine and (+/−)-epibatidine against ³H-(−)-epibatidine to entrapped nAChR in solution and in sol-gel derived macroporous silica. Binding of nicotine and epibatidine to entrapped nAChR indicated by (▪) and (◯) respectively, solid lines indicate fits to the Hill equation. Binding on nicotine and epibatidine to nAChR in solution is indicated by (π) and (♦) respectively, dashed lines indicate fits to the Hill equation.

FIG. 19 shows reusability of nAChR entrapped in macroporous silica as indicated by apparent receptor activity. Light and dark grey bars indicate the difference of unbound ³H-epibatidine with and without the presence of 1.0 mM nicotine. Black bars represent the receptor specific counts, which is the difference between the receptor and liposome unbound counts. Sample regeneration consisted of two 30 min. washes with 100 μL of 500 μM nicotine, followed by six 45 min. washes with 200 μL of assay buffer.

FIG. 20 shows the radioligand binding data for D2R in solution. The receptor specific binding (first bar) and non-specific binding (second bar) values were obtained after subtraction of the values in the filtrate from the total counts added to the sample after accounting for diution effects. Experiments are done in duplicate.

FIG. 21 shows the raw binding data for D2R entrapped in various sol-gel compositions. The compositions and assay conditions are shown in the Figure legend.

FIG. 22 shows the specific D2R binding in macroporous silica obtained by subtracting the non-specific binding data obtained from macroporous silica containing 10% sucrose solution (D2R (specific)=(D2R total−10% sucrose-total)−(D2R non-specific−10% sucrose-with haloperidol).

FIG. 23 shows the binding isotherm obtained for competitive binding of haloperidol against ³H-spiperone to D2R entrapped in a macroporous silica material formed from DGS with 8% PEO. Binding of haloperidol to entrapped D2R is indicated by (♦), solid line indicates fit to the Hill equation.

### DETAILED DESCRIPTION OF THE INVENTION

(i) Method of Entrapping Membrane-Bound Proteins

Phospholipid liposomes with reconstituted ionomycin, gramicidin A or nAChR ion channels were readily incorporated into sol-gel derived silicates without loss of ion channel activity when the sol gel was prepared from protein- and membrane-compatible, silica precursors such as organic polyol-derived silanes and sodium silicate. Steady-state fluorescence measurements of the tryptophan residues of gramicidin A indicated that it remained in its native conformation within the phospholipid membrane following entrapment. It was also found that ion channel activity was retained for reconstituted gramicidin A and that this activity was still sensitive to various electrochemical gradients caused by potassium ion concentration. It was also established that ion flux could be inhibited by the presence of divalent cations; moreover, the ion flux activity through gramicidin channels was retained for several weeks. In addition, it was found that ion channel activity could be produced using either an ionophore (ionomycin) to produce Ca(II) flux across the membrane, or by using the ligand-gated ion channel (LGIC) nAChR, which produced agonist or antagonist dependent transmembrane fluxes of Ca(II). Furthermore, nAChR and the dopamine D2 receptor (D2R, an example of a GPCR) were trapped in a wide variety of sol-gel derived materials (both mesoporous and meso/macroporous) and for the first time the measurement of ligand binding constants for an entrapped membrane-bound protein was determined using conventional radioligand binding assays. A significant improvement in receptor activity (ca. 70% relative to solution) and a significant decrease in non-specific binding was obtained when nAChR or D2R were entrapped into macroporous silicates formed via spinodal decomposition of added PEO (PEO, 10 kDa). Moreover, it was evident that samples retained significant activity upon storage and could be reused.

Accordingly, the present invention relates to a method of immobilizing membrane-associated molecules in silica matrixes comprising combining a liposome assembly comprising the membrane-associated molecule with a protein- and membrane-compatible sol-gel precursor under conditions which allow a gel to form.

Membrane-associated molecules which may be immobilized using the method of the invention include, for example, non-natural ionophores, ion channel proteins, ion-channel receptors, G-protein coupled receptors or membrane associated enzymes. Some specific examples of proteins include gramicidin, bacteriorhodopsin, the nicotinic acetylcholine receptor (aAChR) and the dopamine D2 receptor (D2R). Other examples include: the cys-loop receptor subfamily of LGIC such as GABA_{A}, Glycine, GLUC 1, 5-HT₃ and nicotinic acetylcholine receptors; the ATP gated channel superfamily of LGIC receptors as well as the glutamate cationic receptor superfamily of LGIC receptors; G-protein coupled receptors such as the dopamine and serotonin receptors, histamine receptor and androgenic receptors; membrane transport proteins and membrane associated enzymes such as γ-glutamyltranspeptidase or lipase. Examples of non-natural ionophores, include, for example, various ionophore antibiotics such as ionomycin, monensin, lonomycin, laidlomycin, nigericin, grisorixin, dianemycin, lenoremycin, salinomycin, narasin, antibiotic X206, alborixin, septamycin, antibiotic A204, maduramicin and semduramicin, compound 47224, lasalocid (also including factors A, B, C, D and E), mutalomycin, isolasalocid A, lysocellin, tetronasin, echeromycin, antibiotic X-14766A, antibiotic A23187, antibiotic A32887, compound 51532 and K41 ionomycin, and any other non-natural molecules that act as membrane ion transporters. These lists are not exhaustive, but are meant to provide selected examples of the types of proteins and other membrane-associated molecules that may be used in the current invention. One of ordinary skill in the art would appreciate that other membrane-bound or membrane-associated molecules will also be amenable to the immobilization method described herein.

As used herein, the term “immobilized” means that the liposome assembly is physically, electrostatically or otherwise confined within the nanometer-scale pores of the biomolecule-compatible matrix. In an embodiment of the invention, the assembly does not associate with the matrix, and thus is free to rotate within the solvent-filled pores. In a further embodiment of the invention, the assembly is optionally further immobilized through electrostatic, hydrogen-bonding, bioaffinity, covalent interactions or combinations thereof, between the lipid bilayer and the matrix. In a specific embodiment, the immobilization is by physical immobilization within nanoscale pores.

The term “liposome assembly comprising the membrane-associated molecule” as used herein means that membrane-associated molecule is either extrinsically or intrinsically associated with the lipid components in the liposome though hydrophobic, electrostatic, hydrogen-bonding, bioaffinity, covalent interactions or combinations thereof. The membrane-associated molecule may be associated with the headgroups or acyl chains of the liposome or with both.

The terms “a” and “an” as used herein, unless otherwise indicated, also denotes “one or more”.

By “biomolecule-compatible” and “membrane compatible” it is meant that the silica matrix either stabilizes proteins, membranes and/or other biomolecules against denaturation or does not facilitate denaturation. The term “biomolecule” as used herein means any of a wide variety of proteins, enzymes, organic and inorganic chemicals, other sensitive biopolymers including DNA and RNA, and complex systems including whole or fragments of plant, animal and microbial cells that may be entrapped in the matrix.

In the invention, the biomolecule-compatible and membrane-compatible matrix is a sol-gel. In particular, the sol-gel is prepared using biomolecule- and membrane-compatible techniques, i.e. the preparation involves biomolecule- and membrane-compatible precursors and reaction conditions that are biomolecule- and membrane-compatible. In a further embodiment of the invention, the biomolecule-compatible sol gel is prepared from a sodium silicate precursor solution. In still further embodiments, the sol gel is prepared from organic polyol silane precursors. Examples of the preparation of biomolecule-compatible sol gels from organic polyol silane precursors are described in inventor Brennan and Brook's co-pending patent applications entitled “Polyol-Modified Silanes as Precursors for Silica”, PCT patent application S.N. PCT/CA03/00790, filed on Jun. 2, 2003 and corresponding U.S. patent application filed on Jun. 2, 2003; and “Methods and Compounds for Controlling the Morphology and Shrinkage of Silica Derived from Polyol-Modified Silanes”, PCT patent application WO 04/018360, filed Aug. 25, 2003 and corresponding U.S. patent application filed on Aug. 25, 2003, the contents of all of which are incorporated herein by reference. In specific embodiments of the invention, the organic polyol silane precursor is prepared by reacting an alkoxysilane, for example tetraethoxysilane (TEOS) or tetramethoxysilane (TMOS), with an organic polyol. In an embodiment, the organic polyol is selected from sugar alcohols, sugar acids, saccharides, oligosaccharides and polysaccharides. Simple saccharides are also known as carbohydrates or sugars. Carbohydrates may be defined as polyhydroxy aldehydes or ketones or substances that hydroylze to yield such compounds. The organic polyol may be a monosaccharide, the simplest of the sugars, or a carbohydrate. The monosaccharide may be any aldo- or keto-triose, pentose, hexose or heptose, in either the open-chained or cyclic form. Examples of monosaccharides that may be used in the present invention include one or more of allose, altrose, glucose, mannose, gulose, idose, galactose, talose, ribose, arabinose, xylose, lyxose, threose, erythrose, glyceraldehydes, sorbose, fructose, dextrose, levulose and sorbitol. The organic polyol may also be a disaccharide, for example, one or more of, sucrose, maltose, cellobiose and lactose. Polyols also include polysaccharides, for example one or more of dextran, (500-50,000 MW), amylose and pectin. In embodiments of the invention the organic polyol is selected from one or more of glycerol, sorbitol, maltose, trehelose, glucose, sucrose, amylose, pectin, lactose, fructose, dextrose and dextran and the like. In embodiments of the present invention, the organic polyol is selected from glycerol, sorbitol, maltose and dextran. Some representative examples of the resulting polyol silane precursors suitable for use in the methods of the invention include one or more of diglycerylsilane (DGS), monosorbitylsilane (MSS), monomaltosylsilane (MMS), dimaltosylsilane (DMS) or dextran-based silane (DS). In embodiments, the polyol silane precursor is selected from one or more of DGS and MSS.

In a particular embodiment of the invention, the membrane-associated molecule and sol-gel precursor are combined with an additive which causes spinodal decomposition (phase transition) before gelation, to provide macroporous silica matrixes. Methods of forming macroporous silica, in particular, from polyol-modified silane precursors are described in inventor Brennan and Brook's co-pending patent application entitled “Methods and Compounds for Controlling the Morphology and Shrinkage of Silica Derived from Polyol-Modified Silanes”, PCT patent application WO 04/018360, filed Aug. 25, 2003 and corresponding U.S. patent application filed on Aug. 25, 2003, the contents of which are incorporated herein by reference. In particular, the membrane-associated molecule and sol-gel precursor are combined with one or more water soluble polymers which causes spinodal decomposition (phase transition) before gelation, The water soluble polymer may be selected from any such compound and includes, but is not limited to, for example, polyethylene oxide (PEO); polyethylene glycol (PEG); amino-terminated polyethylene glycol (PEG-NH₂); amino-terminated polyethylene oxide (PEO-NH₂); polypropylene glycol (PPG); polypropylene oxide (PPO); polypropylene glycol bis(2-amino-propyl ether) (PPG-NH₂); polyalcohols, for example, polyvinyl alcohol; polysaccharides; poly(vinyl pyridine); polyacids, for example, poly(acrylic acid); polyacrylamides e.g. poly(N-isopropylacrylamide) (polyNIPAM); or polyallylamine (PAM), or mixtures thereof. In embodiment of the invention the water soluble polymer is selected from PEO, PEO-NH₂, PEG, PPG-NH₂, polyNIPAM and PAM, and mixtures thereof. In further embodiments of the invention, the water soluble polymer is selected from PEO, PEO-NH₂ and polyNIPAM, and mixtures thereof. In still further embodiments, the water soluble polymer is PEO, for example PEO having a molecular weight between about 2000-100000 Da, suitably between about 5000 and 50000 Da, more suitably between about 8000 and 15000 Da. By “water soluble” it is meant that the polymer is capable of being formed into an aqueous solution having a suitable concentration. It should be noted that the terms “oxide” (as in polyethylene oxide) and “glycol” (as in polyethylene glycol) may be used interchangeably and the use of one term over the other is not meant to be limiting in any way.

Macroporous silicas may also be obtained by combining the membrane-associated molecule and sol-gel precursor are with one or more compounds of Formula I: 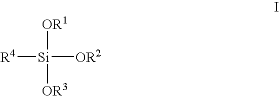
wherein wherein R¹, R² and R³ are the same or different and represent a group that may be hydrolyzed under normal sol-gel conditions to provide Si—OH groups; and R⁴ is group selected from polymer-(linker)ₙ-and 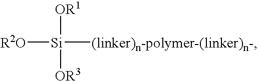
where n is 0 or 1, Such compounds are also described in detail in inventor Brennan and Brook's co-pending patent application entitled “Methods and Compounds for Controlling the Morphology and Shrinkage of Silica Derived from Polyol-Modified Silanes”, PCT patent application WO 04/018360, filed Aug. 25, 2003 and corresponding U.S. patent application filed on Aug. 25, 2003, the contents of which are incorporated herein by reference.

In embodiments of the invention, OR¹, OR² and/or OR³ are the same or different and are derived from organic mono-, di-, or polyols. By “polyol”, it is meant that the compound has more the one alcohol group. The organic portion of the polyol may have any suitable structure ranging from straight and branched chain alkyl and alkenyl groups, to cyclic and aromatic groups. For the preparation of biomolecule compatible silicas, it is preferred for the organic polyol to be biomolecule compatible. In an embodiment of the invention, the groups OR¹, OR² and/or OR³ are derived from sugar alcohols, sugar acids, saccharides, oligosaccharides and polysaccharides. Simple saccharides are also known as carbohydrates or sugars. Carbohydrates may be defined as polyhydroxy aldehydes or ketones or substances that hydrolyse to yield such compounds. The polyol may be a monosaccharide, the simplest of the sugars or carbohydrate. The monosaccharide may be any aldo- or keto-triose, pentose, hexose or heptose, in either the open-chained or cyclic form. Examples of monosaccharides that may be used in the present invention include, but are not limited to allose, altrose, glucose, mannose, gulose, idose, galactose, talose, ribose, arabinose, xylose, lyxose, threose, erythrose, glyceraldehydes, sorbose, fructose, dextrose, levulose and sorbitol. The polyol may also be a disaccharide, for example, but not limited to sucrose, maltose, trehalose, cellobiose or lactose. Polyols also include polysaccharides, for example, but not limited to dextran, (500-50,000 MW), amylose and pectin. Other organic polyols that may be used include, but are not limited to glycerol, propylene glycol and trimethylene glycol. In embodiments of the present invention, the group OR¹, OR² and/or OR³ are derived from a polyol selected from glycerol, sorbitol, maltose, trehalose, glucose, sucrose, amylose, pectin, lactose, fructose, dextrose and dextran and the like. In further embodiments of the present invention, the organic polyol is selected from glycerol, sorbitol, maltose and dextran.

In other embodiments of the invention, OR¹, OR² and OR³ are the same and are selected from C₁₋₄alkoxy, for example, methoxy or ethoxy, aryloxy and arylalkyleneoxy. In further embodiments of the invention, OR¹, OR² and OR³ are all ethoxy. It will be apparent to those skilled in the art that other leaving groups such as chloride or silazane may also be used for the formation of silica according to the methods described in the invention.

The term “aryloxy” as used herein means phenoxy or naphthyloxy wherein, the phenyl and naphthyl groups may be optionally substituted with 1-5 groups, specifically 1-3 groups, independently selected from the group consisting of halo (fluoro, bromo, chloro or iodo), C₁₋₆alkyl, C₁₋₆alkoxy, OH, NH₂, N(C₁₋₆alkyl)₂, NHC₁₋₆alkyl. C(O)C₁₋₆alkyl. C(O)NH₂, C(O)NHC₁₋₆alkyl, OC(O)C₁₋₆alkyl, OC(O)OC₁₋₆alkyl, NHC(O)NHC₁₋₆alkyl, phenyl and the like.

The term “arylalkyleneoxy” as used herein means aryl-(C₁₋₄)-oxy wherein aryl has the same meaning as in “aryloxy”. Specifically, “arylalkyleneoxy” is a benzyl or naphthylmethyl group (i.e. aryl-CH₂—O).

It should be noted that the groups OR¹, OR² and OR³ are capable of participating directly in the hydrolysis/polycondensation reaction. In particular, these functional groups are alkoxy groups attached to the silicon atom at oxygen, i.e., “Si—OR”, which may be hydrolyzed to provide “Si—O—H”, which can condense with other “Si—O—H” or “Si—OR” groups to provide “Si—O—Si” linkages and eventually a three-dimensional network within a gel. Trifunctional silanes form silsesquioxanes upon hydrolysis and there is a lower degree of crosslinking in systems derived therefrom, in particular when compared with systems derived from tetrafunctional silanes. The remaining group attached to the silicon atom (R⁴) is a group that generally does not participate directly in the hydrolysis/polycondensation reaction.

R⁴ is a group that is not hydrolyzed under normal sol-gel conditions and preferably is stabilizing to biological substances, in particular proteins. In specific embodiments, R⁴ is selected from one of the following groups: 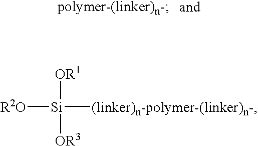
wherein n is 0-1 and OR¹, OR² and OR³ are as defined above. The term “polymer” in R⁴ refers to any water soluble polymer, such as, but not limited to: polyethylene oxide (PEO); polyethylene glycol (PEG); amino-terminated polyethylene glycol (PEG-NH₂); amino-terminated polyethylene oxide (PEO-NH₂); polypropylene glycol (PPG); polypropylene oxide (PPO); polypropylene glycol bis(2-amino-propyl ether) (PPG-NH₂); polyalcohols, for example, polyvinyl alcohol; polysaccharides; poly(vinyl pyridine); polyacids, for example, poly(acrylic acid); polyacrylamides e.g. poly(N-isopropylacrylamide) (polyNIPAM); or polyallylamine (PAM). A linker group is required (i.e. n=1) when a direct bond between the silicon atom and the polymer would be hydrolyzed under normal sol-gel conditions. In embodiments of the invention, the polymer is a water soluble polyether such as PEO.

The sugar and polymer residues may be attached to the silicon atom through any number of linkers. Such linkers may be based on, for example, alkylene groups (i.e. —CH₂)ₘ—, m=1-20, specifically 1-10, more specifically 1-4), alkenylene groups (i.e. —(CH═CH)ₘ—, m=1-20, specifically 1-10, more specifically 1-4), organic ethers, thioethers, amines, esters, amides, urethanes, carbonates and ureas. A person skilled in the art would appreciate that they are numerable linkers that could be used to connect the group, R⁴, to the silicon atom.

Illustrative of compounds of Formula I wherein R⁴ is 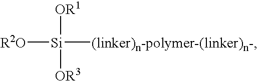
wherein OR¹, OR² and OR³ are as defined above, are compounds 5 shown in Scheme 1. Compounds 5 can be prepared, for example, by reacting poly(ethylene oxide), first with allyl bromide (or any other suitable allylating reagent), followed by reaction with a trialkoxy-, triarylalkyleneoxy- or triaryloxysilane, in the presence of a catalyst, such as a platinum-derived catalyst, as shown in Scheme 1. When modified PEO polymers are used, for example the compound of Formula 5, it is an embodiment of the invention that the starting PEO have a MW of greater than about 2000 g/mol. In this example the linker is an alkylene group, with m=3. Note some allyl-terminated PEO polymers 4 are commercially available. It would be apparent to one skilled in the art that other levels of functionality can also be used to bind these species to the siliceous matrix, such as: R₃₋ₖJₖSi-linker-polymer-linker-SiJₖR₃₋ₖ and polymer-linker-SiJₖR₃₋ₖ where k=1-3 and J is a group that can participate in hydrolysis and condensation with the silica network.

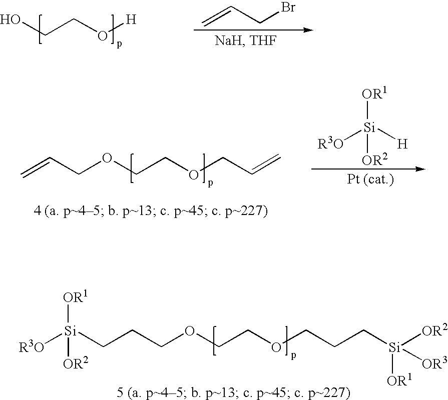

In further embodiments of the invention, the biomolecule-compatible matrix precursor is selected from one or more of functionalized or non-functionalized alkoxysilanes, polyolsilanes or sugarsilanes; functionalized or non-functionalized bis-silanes of the structure (RO)₃Si—R′—Si(OR)₃, where R may be ethoxy, methoxy or other alkoxy, polyol or sugar groups and R′ is a functional group containing at least one carbon (examples may include hydrocarbons, polyethers, amino acids or any other non-hydrolyzable group that can form a covalent bond to silicon); functionalized or non-functionalized chlorosilanes; and sugar, polymer, polyol or amino acid substituted silicates.

In yet another embodiment of the present invention, the biomolecule compatible matrix comprises an effective amount of one or more other additives. In embodiments of the invention the other additives are present in an amount to enhance the mechanical, chemical and/or thermal stability of the matrix and/or assembly components. In an embodiment, the mechanical, chemical and/or thermal stability is imparted by a combination of precursors and/or additives, and by choice of aging and drying methods. Such techniques are known to those skilled in the art. In further embodiments of the invention, the additives are selected from one or more of humectants and other protein stabilizing agents (for e.g. osmolytes). Such additives include, for example, one or more of organic polyols, hydrophilic, hydrophobic, neutral or charged organic polymers, block or random co-polymers, polyelectrolytes, sugars (natural or synthetic), and amino acids (natural and synthetic). In embodiments of the invention, the one or more additives are selected from one or more of glycerol, sorbitol, sarcosine and polyethylene glycol (PEG). In further embodiments, the additive is glycerol.

In a particular embodiment of the invention biocompatible matrix is a silica based glass prepared from, for example, a silicon alkoxide, alkylated metal alkoxide or otherwise functionalized metal alkoxide or a corresponding metal chloride, silazane, polyglycerylsilicate, diglycerylsilane or other silicate precursor, optionally in combination with additives selected from one or more of any available water soluble polymers, compounds of Formula I, organic polymers, polyelectrolytes, sugar (natural or synthetic) or amino acids (natural and non natural). The preparation of sodium silicate solutions for use as a sol-gel precursor is known in the art.³⁸ The use of sodium silicate as a sol-gel precursor may be problematic if either sodium or potassium ions are to be transported through the membrane of the liposome due to interference from the sodium ions present in the precursor solution. In these circumstances, it is preferred to use the organic polyol silane precursors described above. In the case of ligand-gated ion channels, the sodium may not enter the internal compartment of the liposome in the absence of ligand, accordingly the residual sodium could be washed away before use allowing sodium silicate to be a suitable precursor for the transport of sodium or potassium ions through these types of membrane associated molecules.

The liposome-molecule assembly can be prepared using methods known to-those skilled in the art. Typically a solution of the membrane-associated molecule, either with or without its intrinsic lipids (if any) present, is combined with a solution of a suitable lipid. Any lipid which forms liposomes may be used, for example, phospholipids, such as 1,2-dioleoyl-sn-glycero-3-phosphocholine (DOPC). Suitable lipid components may include, but are not limited to: phospholipids, sphingolipids, glycolipids, synthetic and non-natural lipids, fluorescently labelled lipids, polymer-linked and polymerizable lipids (i.e., diacetylenic lipids), photoreactive lipids, fatty acids, fatty amines and hydrophobic moieties such as cholesterols, sterols etc. These may be used alone or in combination, and the resulting liposomes may contain mixtures of single or double chain surfactants, with chain lengths in the range of 4-30 carbons, with between 0 and 10 sites of unsaturation per chain. Upon formation of the lipid mixture, all organic solvents are removed (if necessary) and the resulting lipid films may be rehydrated in suitable buffer solutions followed by conversion to lipid vesicles (by sonication and/or extrusion, or any other suitable method) with the membrane associated molecule embedded within the lipid bilayer.

The liposome-molecule assembly may be combined with a protein- or membrane-compatible, sol-gel precursor solution under conditions which allow a gel to form. By “gel” it is meant a solution or “sol” that has lost flow. The sols lose flow due to the hydrolysis and polycondensation of the precursor. The hydrolysis and condensation of the polyol silane and sodium silicate precursors may suitably be carried out in aqueous solution. Suitably, a solution, for example a homogeneous solution, of precursor, in acidified water is used, or in the case of DGS a solution of the precursor in water or buffer at neutral pH. Sonication may be used in order to obtain a homogeneous solution. By “homogeneous” it is meant having an essentially uniform composition or structure. Conditions which allow the formation of a gel comprise adjusting the pH of the aqueous solution of precursor so that formation of a gel occurs. Suitably, the pH may be in the range of about 4-11.5. The pH may be adjusted, for example, by the addition of suitable buffer solutions or resins. As the solutions lose flow, they can be formed, cast, moulded, shaped, spun, pin-printed as microarrays or drawn into desired shapes. Examples of such shapes include, but are not limited to, films, fibres, monoliths, pellets, granules, tablets, rods or bulk. The solutions may also be placed into multi-well plates for high-throughput screening applications, or printed as microarrays for multianalyte sensing or screening. Accordingly, in an embodiment of the present invention, the method of immobilizing membrane-associated molecules in silica matrixes comprises:
(i) combining an aqueous solution of the protein- and membrane-compatible, sol-gel precursor with an aqueous solution of a liposome assembly comprising the membrane-associated molecule;
(ii) adjusting the pH of the combination of (i) so that it is in the range of about 4-11.5;
(iii) shaping the combination into a desired shape;
(iv) allowing the combination to gel; and
(v) aging and partially drying the gel.

A person skilled in the art would appreciate that the conditions may need to be adjusted depending on the identity of the sol-gel precursor and the liposome assembly and could do so without undue experimentation in light of the present disclosure and the examples provided herein.

Once the gel has been formed and shaped it may be aged over a period of time under select conditions to lock the conformation of the gel, its pores, matrixes and interconnecting channels into fixed positions and permit long term storage. In embodiments of the invention, the gels are aged in buffer or in a solution comprising an effective amount of a humectant, for example glycerol (suitably about 5-50% (v/v) of glycerol in water or buffer solution, preferably 25% (v/v) of glycerol in water or buffer solution).

In embodiments of the invention, the protein- and membrane-compatible, sol-gel precursor solution and the liposome assembly are combined in the presence of an indicator molecule. Alternatively, the liposome assembly further comprises an indicator molecule located on the interior of the liposome, provided that the agonist/antagonist does not have specific interactions with the fluo-3 dye.⁵⁸ As used herein, the term “indicator molecule” refers to any compound that may be used to detect a change in the membrane-associated molecule's conformation or activity, including trans-membrane ion fluxes. Examples of such indicator molecules include compounds which have at least one detectable characteristic which is sensitive to changes in, for example, pH, membrane potential, ionic strength, divalent ion concentration or the hydrophilicity/hydrophobicity of its environment. Specific examples of such an indicator molecules are the lipophilic cationic dye safranine 0, the fluorescence of which is sensitive to changes in membrane potential, and the fluorescent dye fluo-3, which is sensitive to the concentration of free Ca(II) in solution.

In embodiments of the invention, the protein- and membrane-compatible, sol-gel precursor solution and the liposome assembly are combined in the presence of one or more ligands (natural or unnatural) for the protein (for example a receptor) in question, that may optionally be labelled, for example, fluorescently labelled, for detection of activity of the protein. The term “label” refers to any detectable moiety. A label may be used to distinguish a particular ligand from others that are unlabelled, or labelled differently, or the label may be used to enhance detection.

Herein, the entrapment of the reconstituted ion channel peptide gramicidin A (gA) into sol-gel derived silica is reported along with the measurement of ion flux through the membrane using a novel fluorescence method based on the potential-sensitive probe Safranine O. Gramicidin A was chosen as a model system since the fluorescence properties of the tryptophan residues of gA can be measured to determine protein conformation and local environment in solution and upon reconstitution and entrapment.^{54,55,56,57} The results clearly demonstrate that upon entrapment gramicidin remains embedded in the phospholipid membrane and that its ion channel activity is retained upon entrapment.

The entrapment of a reconstituted ionophore (ionomycin) and a ligand-gated transmembrane receptor (ACHR) into sol-gel derived silica is also reported along with the measurement of Ca(II) ion flux through the membrane using a novel fluorescence method based on the Ca(II)-sensitive probe fluo-3. Ionomycin was chosen as a model system since the ionophore can be used to produce pores in the membrane to optimize fluorescence signals resulting from Ca(II) transmembrane ion flux. AChR was chosen as it is a pharmacologically relevant ligand-gated receptor that has the potential to be used as a drug target. The results clearly demonstrate that upon entrapment the AChR remains embedded in the phospholipid membrane and that its ion channel activity is retained upon entrapment.

The present invention further relates to protein- and membrane-compatible sol-gels with a liposome/membrane-associated molecule assembly immobilized therein and prepared using the method as described hereinabove.

(ii) Uses

The immobilization of membrane-associated molecules is important in several technologies including the development of biosensors, protein microarrays and bioaffinity columns. The sol-gels prepared using the method described in the previous section can be used for any of these applications. In particular, the gels may be used to screen for agonists, antagonists and modulators of any membrane associated molecule, such as non-natural ionophores, ion-channel receptors, G-protein coupled receptors or membrane-associated enzymes; microarraying of protein-membrane complexes for high-throughput screening of modulators of membrane-bound receptors; or immobilization of membrane-bound receptors into sol-gel derived monolithic columns for drug screening by frontal-affinity chromatography with mass spectrometric detection.

Accordingly, also included within the scope of the present invention are methods for the detection of modulators of a membrane-associated molecule comprising:
(a) exposing a liposome assembly comprising the membrane-associated molecule, said assembly being immobilized in a protein- and membrane-compatible sol-gel, to one or more test substances; and
(b) detecting a change in one or more characteristics of the membrane associated molecule.
   In embodiments of the invention, the protein- and membrane-compatible sol-gel is prepared using a method described herein. In further embodiments of the invention, a change in the one or more characteristics of the membrane-associated molecule in the presence of the one or more test substances compared to a control indicates that the one or more test substances are modulators of the membrane-associated molecule.

By “control” is meant repeating the same method, under the same conditions but in the absence of the one or more test substances.

The one or more test substances can be any compound which one wishes to test including, but not limited to, proteins (including antibodies), peptides, nucleic acids (including RNA, DNA, antisense oligonucleotide, peptide nucleic acids, RNA or DNA aptamers, ribozymes or deoxyribozymes), fragments of proteins, peptides, and nucleic acids carbohydrates, organic compounds, inorganic compounds, natural products, library extracts, bodily fluids and other samples that one wishes to test for modulators of the membrane-bound protein. The one or more test substance may be in liquid or gaseous form. Typically a solution of known concentration of the one or more test substances is employed.

In embodiments of the invention, the method for the detection of modulators of a membrane-associated molecule further involves a liposome assembly comprising a membrane-associated molecule in combination with other entities that facilitate the detection of modulation of the membrane-associated molecule by the one or more test substances. In an embodiment of the invention the other entities are selected from one or more of indicator molecules and ligands (natural or unnatural) for the receptor protein being investigated. In embodiments of the invention, the ligands may be labelled or unlabelled.

The method of detecting modulators of membrane-associated molecules may be “miniaturized” in an assay system through any acceptable method of miniaturization, including but not limited to multi-well plates, such as 24, 48, 96 or 384-wells per plate, microfluidic chips, microarrays or slides. The assay may be reduced in size to be conducted on a microfluidic-chip support, advantageously involving smaller amounts of reagents and other materials. Any miniaturization of the process which is conducive to high-throughput screening is within the scope of the invention. The “one or more characteristics of the membrane-associated molecule” that may be used to detect modulators of the membrane-associated molecule include, but are not limited to, molecule-mediated transmembrane ion fluxes and conformational/environmental changes in the protein, membrane or a probe molecule that is associated with the protein or membrane, or entrapped within the liposome, or by binding of fluorescent or radioactive ligands by the entrapped protein.

In an embodiment of the invention the membrane-associated molecule is an ion channel protein or ionophore and the characteristic of the membrane-associated protein or ionophore that is detected is the flux of ions through the protein or ionophore from the exterior of the liposome to the interior. Such a flux or movement of ions results in the formation of an electrochemical potential across the liposome membrane and/or in the presence of a specific ion within the liposome. Certain fluorescent indicator molecules, for example the lipophilic cationic dye safranine O, respond to the development of membrane potential by partitioning to certain locations in the assembly resulting in either an increase or decrease in fluorescence intensity and anisotropy. Other dyes, such as fluo-3, respond to the presence of specific ions, such as Ca(II), resulting in a large increase in fluorescence intensity. Modulation of this change in fluorescence intensity and/or anisotropy by the one or more test substances can be used as a means to detect modulators of membrane-associated molecules.

In a further embodiment of the present invention, the membrane associated molecule is a membrane receptor, for example a G-protein coupled receptor, such as D2R, and the characteristic of the membrane-associated molecule that is detected is binding to a ligand, for example a radiolabelled ligand. In further embodiments of the invention, the sol-gel entrapped membrane receptors can be used in standard radioligand displacement binding assays to identify other substances that bind to the receptor. In such assays, it is suitable for the membrane associated molecule to be entrapped in macroporous silica.

In an embodiment of the invention, the sol-gel entrapped liposomes comprising membrane associated molecules are formed into microarrays. Microarrays may be formed by pin-printing the solution comprising the liposome assembly and the sol-gel precursors onto a suitable surface in array format before the solution gels. The solutions are then allowed to gel and dry on the surface. Suitable methods for forming sol-gel microarrays are known in the art (see, for example, inventor Brennan's co-pending PCT Patent Applicant S.N PCT/CA03/01665 and U.S. regular application Ser. No. 10/698,492, entitled “Multicomponent Protein Microarrays”, filed on Nov. 3, 2003). The present invention provides the first example of the use of transmembrane ion flux as a signalling method for microarrays.

Fluorescence is only one of many means of detecting change in one or more characteristics of the membrane-associated molecule. Because of the light-transmission capabilities of the matrixes of the present invention, UV, IR and visible light optical spectroscopy, as well as luminescence, adsorption, emission, excitation and reflection techniques are all suitable for detecting changes in the characteristics of the entrapped membrane associated molecule.

The present invention also includes kits, biosensors, microarrays, chromatographic and bioaffinity columns comprising the silica matrixes comprising a liposome-protein assembly prepared as described herein. The kits of the present application comprise, in different combinations, the matrixes, reagents for use with the matrixes, signal detection and processing instruments, databases and analysis and database management software above. The kits may be used, for example, to determine the effect of one or more test compounds on a membrane-associated molecule and to screen known and newly designed drugs.

Yet another aspect of the present invention provides a method of conducting a target discovery business comprising:
(b) providing one or more assay systems for identifying test substances by their ability to effect one or more membrane-associated molecules based systems, said assay systems using a method of the invention;
(b) (optionally) conducting therapeutic profiling of the test substances identified in step (a) for efficacy and toxicity in animals; and
(c) licensing, to a third party, the rights for further drug development and/or sales or test substances identified in step (a), or analogs thereof.
   (iii) Improved Method for Detecting Membrane Potentials

A novel procedure amenable to the sol-gel method of entrapment was developed to monitor ion flux through an entrapped membrane-associated molecule. In this method, the fluorescence indicator used to detect the development of a potential across the lipid membrane or the presence of a specific ion inside the liposome due to ion flux was located on the inside of the liposome assembly only. Literature methods describe the use of fluorescent indicators in the external solution. Problems caused by interactions of the indicator molecule with the anionic surface of the silica can arise when the indicator molecules are in the external solution. Such problems are avoided when the indicator molecules are located within the interior of the liposome assembly since in this location these molecules are not able to interact with the silica surfaces. Accordingly, the present invention further relates to a method for preparing a sol gel immobilized liposome assembly comprising a membrane associated molecule, wherein the membrane-associated molecule is an ion-channel molecule, comprising:
(a) obtaining a solution of the liposome assembly having an indicator molecule located on the interior of the assembly;
(b) removing the indicator molecule from solution external to the liposome assembly; and
(c) combining the liposome assembly solution with a silica precursor solution under conditions which allow a gel to form.

The present invention also relates to an improved method for the detection of membrane potentials in a sol-gel immobilized liposome assembly comprising a membrane-associated molecule, wherein the membrane-associated molecule is an ion-channel molecule, comprising:
(a) obtaining a solution of the liposome assembly having an indicator molecule located on the interior of the assembly;
(b) removing the indicator molecule from solution external to the liposome assembly;
(c) combining the liposome assembly solution with a silica precursor solution under conditions which allow a gel to form;
(d) contacting the gel with the ion and optionally a test substance; and
(e) detecting a change in the indicator molecule upon transmembrane ion flux.

In embodiments of the invention the indicator molecule can be any compound that interacts with the surface of the sol gel, for example, the lipophilic cationic dye, safranine O. In further embodiments, the indicator molecule acts by detecting the ion directly upon entry into the interior of an entrapped liposome, for example the calcium dependent fluorophore, fluo-3, provided that the agonist/antagonist does not have specific interactions with the fluo-3 dye.⁵⁸ In still further embodiments, the indicator molecule is removed from the solution external to the protein-liposome assembly using dialysis or gel filtration chromatography.

In embodiments of the invention, the silica precursor is biomolecule- and membrane-compatible. In still further embodiments, the liposome assembly further comprises a ligand (natural or unnatural, labelled or unlabelled) for the membrane associated molecule (for example a receptor).

The following non-limiting examples are illustrative of the present invention:

### EXAMPLES

Materials

1,2-Dioleoyl-sn-glycero-3-phosphocholine (DOPC), Egg phosphatidylcholine (EggPC), Egg Phosphatidylethanolamine (EggPE) and sphingomylin were purchased from Avanti Polar-Lipids, Inc (Alabaster, Ala.). Gramicidin A, High Purity (95%) was purchased from Calbiochem (San Diego, Calif.). The human nicotinic acetylcholine receptor was purchased from Perkin Elmer Life Sciences (Boston, Mass.) while Torpedo californica nAChR was purified from the electric organ of the organism according to established protocols.⁵⁹ Diglyceryl silane (DGS) was provided by Dr. Michael Brook of McMaster University and was prepared by a method that is described elsewhere.⁶⁰ The fluorescent dye Fluo-3 was purchased from Molecular Probes (Eugene, Oreg.). The fluorescent dye safranine O, sodium silicate solution, Sephadex G25, sucrose, ethylenendiaminetetraacetic acid (EDTA), SM-2 Biobeads, Pottasium phosphate, ethylenediaminetetraacetic acid (EDTA), ethyleneglycolamine-tetraacetic acid (EGTA), potassium chloride, sodium aside, ionomycin, iodoacetamide, (−)-nicotine, cytisine, d-tubocurarine, phenylmethanesulfonylfluoride (PMSF), N-dodecyl-b-D-manopyranoside (DMM), Asolectin® and polymethacrylate fluorimeter cuvettes (transmittance curve C) were obtained from Sigma (St. Louis, Mo.). Black and transparent ninety-six well microwell plates were purchased from Nalge Nunc International (Rochester, N.Y.). ³H-epibatidine was purchased from Amersham Biosciences (Buckinghamshire, UK). Torpedo californica electroplax was purchased from Aquatic Research Consultants (San Pedro, Cal.). Dialysis Tubing with a molecular weight cut-off of 3500 Da was purchased from Spectrum Laboratories Inc. (Rancho Domingez, Calif). All water was twice distilled and deionized to a specific resistance of at least 18 MΩ.cm using a Milli-Q Synthesis A10 water purification system. All other chemicals were of analytical grade and were used without further purification.

Methods

Preparation of Fluo-3 Loaded Liposomes

DOPC stock solutions were purchased in chloroform at a concentration of 20 mg.mL⁻¹. DOPC stock solution was dispensed in disposable glass vials and the organic solvent was removed by evaporation under a dry nitrogen gas stream to remove the bulk of the organic solvent, followed by evaporation under vacuum for two hours. The resulting dried lipid films were then rehydrated in a buffer consisting of 25 mM EGTA, 25 mM EDTA, 10% Sucrose (w/w), 10 mM KCl, 0.529 mM Fluo-3, pH 7.6 to a final lipid concentration of 2 mg/mL. The hydrated liposomes were then extruded through 600 nm pores, with an Avanti—MINIEXTRUDER at room temperature to create a mono-disperse suspension of unilamellar liposomes 600 nm in diameter. The external Fluo-3 was removed by filtration through a column packed with Sephadex G25 to yield liposomes with dye only on the interior of the liposome.

Preparation of Reconstituted Gramicidin:

The Gramicidin A stock was prepared in a solution of chloroform, trifluoroethanol and dimethylsulfoxide (19:5:1 volume ratio) to a final concentration of 4.79×10⁻⁴M. Gramicidin A stock solutions were mixed with the lipid stock solutions (20 mg.mL⁻¹ DOPC in chloroform) in disposable glass vials to provide final ratios of gA:lipid of either 0.39 mol % or 0.94 mol %. The organic solvent was removed by evaporation under a dry nitrogen gas stream to remove the bulk of the organic solvent, followed by evaporation under vacuum for two hours. The resulting dried gA:lipid films were then rehydrated in an appropriate buffered solution followed by high frequency sonication for one hour with a VWR Scientific Aquasonic Model 50T sonicator to create small unilamellar phospholipid vesicles with reconstituted Gramicidin A ion channels.^{61,62,63}

For ion-channel studies, reconstitution of gramicidin A samples was done using an unbuffered aqueous solution containing 50 μM safranine O at pH 7.0. To remove the safranine O from the exterior of the liposomes, samples were dialyzed in distilled deionized water until negligible safranine O fluorescence was observed in the dialysate. Liposome solutions that were used for tryptophan fluorescence studies consisted of 0.94 mol % of gramicidin A in DOPC that was hydrated to final concentrations of 1.0 μM of gA in 100 mM phosphate buffer at pH 7.0.

Preparation of Liposomes Containing Nicotinic Acetylcholine Receptor (nAChR)

Lipid films consisting of EggPC, EggPE, Sphingomylin and cholesterol in mol ratios of 55:27:9:9 percent, respectively, were prepared as described earlier, and rehydrated to a final lipid concentration of 3.0 mg/mL and Fluo-3 concentration of 65 μM. The liposome stock was then mixed with a small amount of DMM bringing the solution to a final concentration of 0.2 mM. Immediately following this, IMR-32 nAChR stock was added to the detergent lipid mixture and allowed to incubate at 4° C. for 90 min. Following this 15 mg of SM2-Biobeads were added to the mixture and incubated for 1 hr. This was repeated 3 times with the final addition allowed to incubate for a period of 12 hrs. The nAChR containing liposomes were then passed through a Sephadex G25 column to remove any additional detergent as well as the extraliposomal Fluo-3. The IMR-32 nAChR containing liposomes were used for ion flux studies without further modification.

Entrapment of Reconstituted Gramicidin:

Diglyceryl silane (DGS) derived sol-gels were prepared by adding 0.212 g of solid DGS and 5 μL of 0.1 N HCl to 650 μL of distilled deionized water followed by sonication at 0° C. for 1.5 hours until all of the silane precursor had been dissolved and the solution had become homogeneous and transparent. Samples used for the collection of Trp spectra were prepared by placing 70 μL of the solution in the well of a microwell plate. For ion channel studies, the dialysed liposomes were mixed in a 1:1 volume ratio with DGS, along with 2 μL of 1M NaOH and 4 μL of 2.5 M NaCl in a microwell plate to a final volume of 76 μL. Sodium hydroxide and sodium chloride were added only to allow gelation to occur, and were not present at a sufficient concentration to produce a significant effect on the flux of potassium ions across the membrane. For Safranine O fluorescence anisotropy studies, thin silica films were prepared by mixing the hydrolyzed DGS precursor solution with the liposome solution in a 1:1 volume ratio to a final volume of 100 μL. The solution was then spin-cast onto a glass slide (8×32 mm) for 1 min at a rate of 2000 rpm. In all cases, the samples were allowed to gel and were then aged in air (dry-aged), in buffer (wet-aged) or in a 25% solution of glycerol in water (glycerol-aged) for 1 to 28 days before fluorescence measurements were done.

Entrapment of DOPC Liposomes and Reconstituted nAChR:

Sodium silicate or DGS derived sol precursors were prepared by methods described previously.⁶⁴ The sol solution was mixed 1:1 (v/v) with the buffered liposome or reconstituted nAChR solution, in the bottoms of 96 well microtiter plates to a final volume of 100 μL. For entrapment of IMR-32 cells for ion flux assays, the nAChR containing stock described above was uses for entrapment without further dilution. For radioassays using IMR-32 nAChR, the stock sample as provided directly from Perkin Elmer was diluted four fold in 25 mM HEPES, 100 mM KCl, 5 mM EGTA, pH 7.4 and mixed 1:1 (v/v) in DGS derived sol. The samples were aged for 1 hr at 4° C. For Torpedo Californica nAChR, the stock sample was diluted four fold in 150 mM HEPES, 100 mM KCl, 5 mM EDTA, pH 7.4, and mixed 1:1 (v/v) with sodium silicate derived sol.

Steady State Tryptophan-Fluorescence Measurements of gA:

Fluorescence measurements were performed using a Jobin Yvon-SPEX Fluorolog-3 Model 212 T-format spectrofluorimeter (ISA Instrument Int. Edison, NJ) with a MicroMAX 96-well fluorescence plate-reader attachment that was interfaced to the spectrofluorimeter using a bifurcated fused silica optical fiber. Tryptophan emission spectra of reconstituted Gramicidin A were collected from 310 to 450 nm using an excitation wavelength of 280 nm. All spectra were collected in 0.5 nm increments using 5 nm bandpasses on the excitation and emission monochromators and an integration time of 0.5 s per point. Appropriate blanks were subtracted from each sample and the spectra were corrected for the wavelength dependence of the emission monochromator and photomultiplier tube.

Ion-Channel Activity Assay of gA:

The fluorescence intensity response of safranine O was monitored using the MICROMAX microwell plate reader. Fluorescence emission was monitored as a function of time at 565 nm with an excitation wavelength of 528 nm upon addition of 125 μL of various concentrations of potassium iodide to the top of the monolithic samples in the microwell plate to create an ionic gradient across the membrane. Emission measurements were performed over a period of 125 s (solution experiments) or 600 s (for entrapped gA) using 0.25 sec intervals with a 0.20 sec integration time and emission and excitation bandpasses of 5 nm. The responses were normalized to the intensity value obtained before addition of the salt solution. Alternatively, fluorescence anisotropy was monitored in the T-format in 1 second intervals with a 0.95 s integration time over a period of 125 s after addition of KI solutions. All anisotropy measurements were done using glass slides with spin-coated films of silica which were mounted at an angle of 55° with respect to the excitation beam (90° geometry). All anisotropy measurements were corrected for the instrumental G factor to account for any polarization bias in the monochromators.

Radioassays of nAChR:

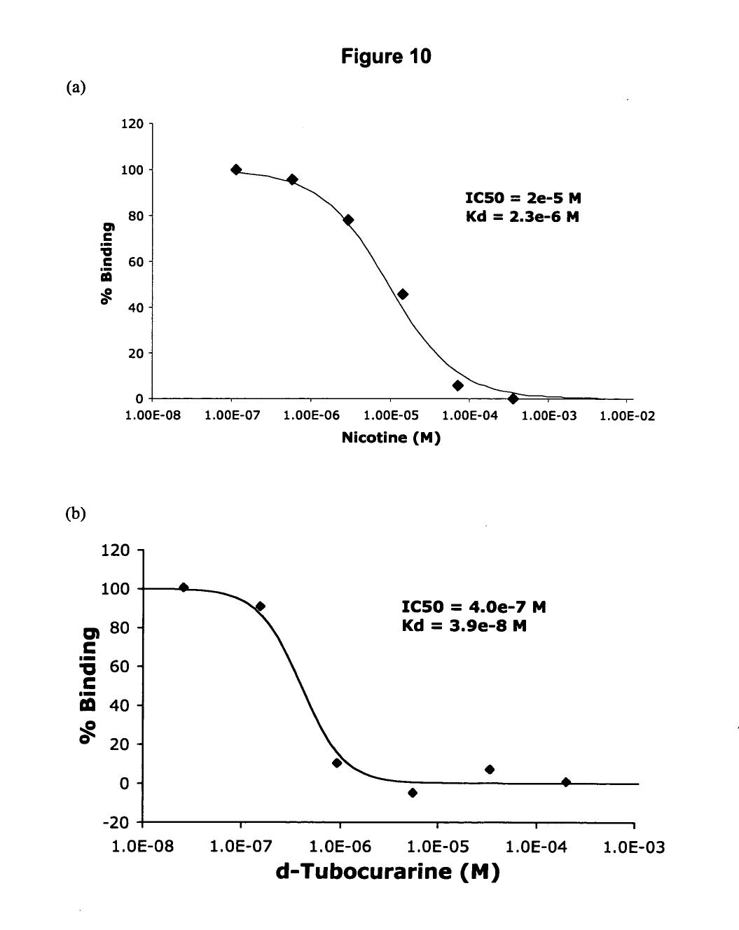

Samples of either entrapped Torpedo californica nAChR, IMR-32 nAChR or Asolectin liposomes were formed in the bottoms of 96-well microtiter plates. 100 μL of nAChR stock (3.72 nmol/mg protein Torpedo californica nAChR or 168 fmol/mg IMR-32 nAChR) or 20 mg/mL Asolectin liposomes were mixed with an equal volume of DGS or sodium silicate sol in the well of a microwell plate, where formation of a solid gel commenced. The monoliths were allowed to cure for 1 hour, following which 10 μL of either buffer or 10 mM nicotine was added and allowed to incubate at 4° C. for 2.5 hrs. 160 μL of ³H-epibatidine in buffer was added to the monoliths to a final concentration of 1.0-3.0 nM, and incubated for 18 hr at 4° C. After incubation, 155 μL of ³H-epibatidine solution was drawn off the top of the monolith and dissolved in 20 mL of Liquiscint scintillation fluid. The radioactive decay from ³H-epibatidine was then counted for 5 min to determine the ratio of free ligand existing in solution. Nicotine was added to determine the amount of specific binding to the receptor itself, and the Asolectin liposome samples were used to evaluate the amount of non-specific binding to the matrix. Using the information from these samples the amount of receptor bound ligand could be determined. For competitive assays seen in FIGS. 10A and 10B, 10 μL various concentrations of either (−)-nicotine or d-tubocurarine were added to the tops on the DGS monoliths containing IMR-32 nAChR or Asolectin liposomes and allowed to incubate for 2.5 hrs. 160 μL of 3.0 nM ³H-epibatidine was then added and the samples were incubated for 18 hrs. Free ligand was determined as described above.

Ion-Channel Activity of IMR-32 nAChR

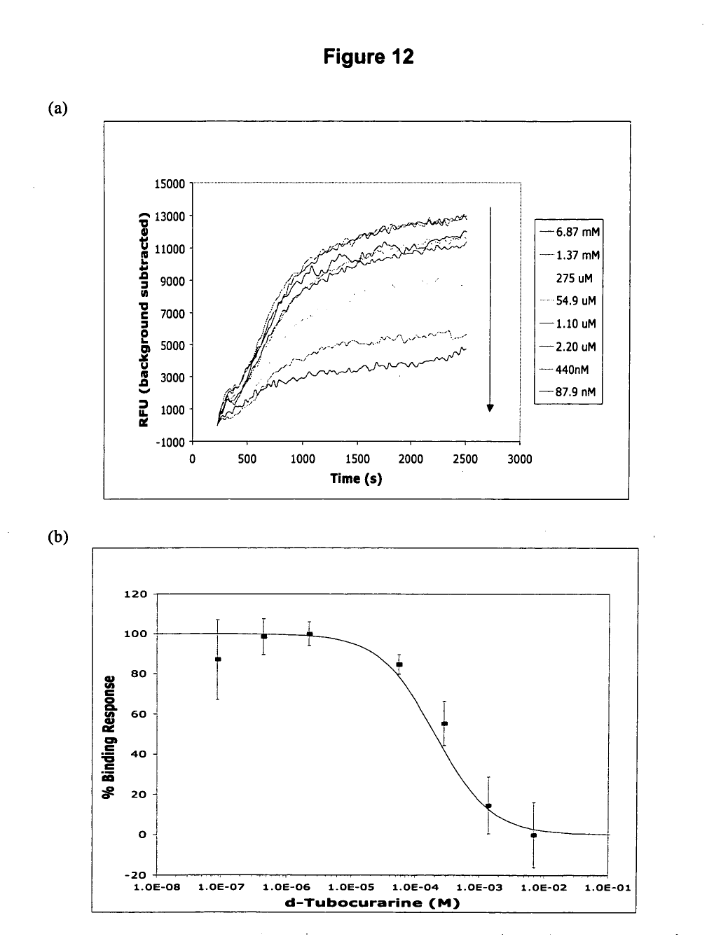

IMR-32 nAChR was used for the Fluo-3 based assays due to its increased calcium permeability as compared to the nAChR derived from Torpedo californica. In these assays, IMR-32 nAChR containing liposomes with an intraliposomal solution of Fluo-3 were entrapped as described above in 1:1 (v/v) in DGS derived silica. The buffered sol was then dispensed in the bottoms of standard 96-well microwell plates and allowed to cure for 1 hr at 4° C. Antagonism of the nAChR ion channel was measured by addition of 25 μL of the nAChR antagonist d-tubocurarine to the top of the nAChR-containing monolith in the 96-well plate. Ligand-gated ion-flux was monitored through time dependent changes in fluorescence intensity upon the addition of 50 μL of 3 M CaCl₂ using a TECAN-Safire microwell platereading fluorescence system. Fluo-3 emission was monitored and 526 nm with an excitation wavelength of 488 nm, emission and excitation bandpasses of 5 and 7.5 nm, and a detector gain of 130 V, over a period of 45 min. Similarly, agonism of the nAChR ion-channel was monitored using the same assay except the channel was first antagonized by incubating the samples in 0.012 M d-tubocurarine, which was then followed by the addition of various concentrations of (−)-cytisine along with 3 M CaCl₂. The time dependent responses were then normalized as a function of their initial fluorescence intensity before the addition of calcium as seen in FIG. 12. The normalized changes in fluorescence intensity for the various concentrations of agonist or antagonist were then scaled as a percentage between their maximum and minimum response and an apparent dissociation constant could be determined by fitting the response to the “Hill” equation.

Microarray Experiments:

Sodium silicate derived sol precursors were prepared by methods described previously.⁶⁵ The sol solution was mixed 1:1 (v/v) with the buffered solution of ionomycin doped DOPC liposomes in the bottoms of 96 well microtiter plates to a final volume of 80 μL.

A Virtek Chipwriter Pro (Virtek Engineering Sciences Inc., Toronto, ON) robotic pinspotter equipped with a SMP 3 Stealth microspotting pin (Telechem Inc., Sunnyvale, Calif.) was used to print the ionmycin:liposome samples onto glass microscope slides from 96-well plates. Printing temperature was ambient with a humidity of approximately 50-70%. Completion of an array of 25 spots (5×5) took about 1 minute to perform, including pin wash cycles when using a printhead speed of 16 mm/s. Fluorescence images of the microarrays were taken with an Olympus BX50 Microscope equipped with a Roper Scientific Coolsnap Fx CCD camera using a multi-line argon ion laser source for excitation of fluo-3 (488 nm). Arrays containing the ionomycin doped DOPC liposomes were imaged before and 1 minute after addition of 1 mM Ca(II) using a 30 second integration time per image.

### EXAMPLE 1

### Tryptophan Fluorescence of gA

The emission of Trp residues within proteins has been widely used to probe the conformation and dynamics of proteins within sol-gel derived silica.^{66,67} In the case of gramicidin A, each homodimeric subunit of the ion channel contains four tryptophan residues, which NMR and crystallographic data have shown to be buried within the lipid bilayer.⁶⁸ Furthermore, the tryptophan residues of gramicidin have been shown to have distinctly different fluorescence emission spectra when located in the bilayer relative to being in solution.⁵⁴ The fluorescence emission properties of gA can therefore be used to indicate if gramicidin has survived the entrapment process and remained in the bilayer.

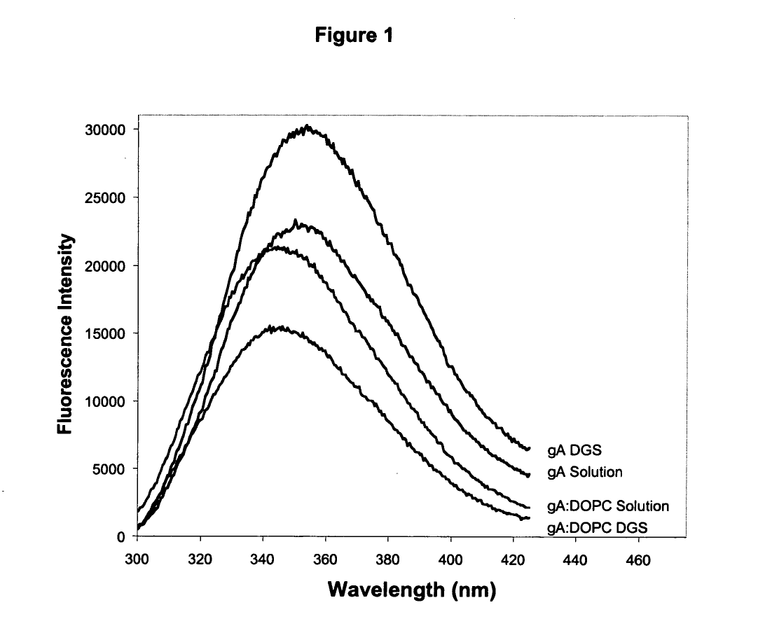

FIG. 1 shows the emission spectra of gramicidin A before and after reconstitution into phospholipid vesicles comprised of DOPC, both in solution and after entrapment into DGS derived silicate. The results clearly show that the emission maximum of gramicidin embedded in DOPC liposomes stays constant at 340 nm in solution and in DGS derived silicate; whereas gramicidin in the absence of liposomes is red-shifted, with a peak emission intensity at 350 nm both in solution and when entrapped. These results show that the gA remains within the bilayer structure when entrapped into DGS derived silicate.

It should be noted that attempts to entrap reconstituted gA into TEOS derived materials were unsuccessful, and generally led to fluorescence spectra that were consistent with aggregation of the gA peptide, and also produced a system that was not able to generate ion fluxes (results not shown). This is likely due to the loss of bilayer lipid membrane integrity resulting from the presence of ethanol,³⁸ which is a byproduct of the hydrolysis of TEOS. On the other hand, the use of the diglyceryl silane precursor, which liberates glycerol as a byproduct of hydrolysis, was able to retain the emission properties of reconstituted gA upon entrapment, and as discussed below, also provided an environment that was conducive to maintaining the ion-channel activity of entrapped gA.

### EXAMPLE 2

### Ion Channel Activity of gA

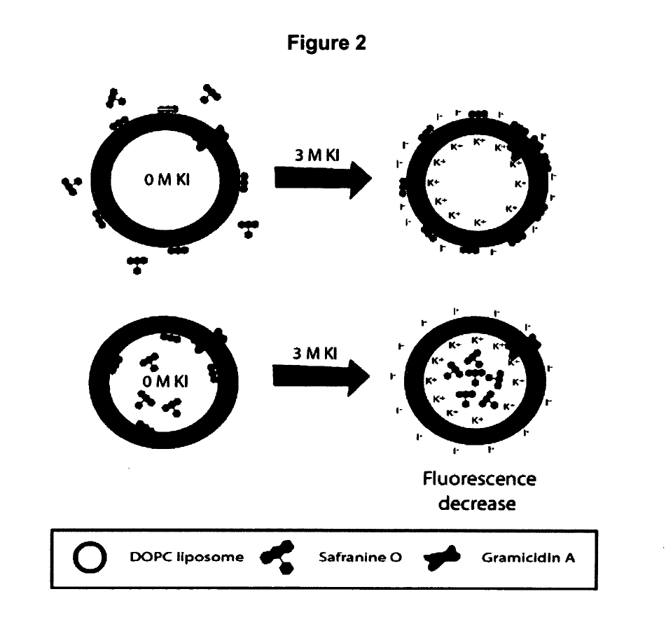

The lipophilic cationic dye safranine O was used to follow the development of an electrochemical potential of K⁺ across the phospholipid membrane. As shown in FIG. 2, the changes in emission properties depend on whether the probe is located inside or outside of the membrane. As shown in FIG. 2a, upon addition of KCl or KI to a membrane with the probe in the external solution, the influx of potassium ions through gA into the interior of the liposomes, combined with the exclusion of chloride ions, creates an electrochemical gradient across the membrane that is net positive on the interior and net negative on the exterior. Safranine O responds to development of such a membrane potential by partitioning into the hydrophobic lipid core due to the electrostatic attraction of the dye to the net-negative side of the membrane.^{69,70,71} The net effect is to produce an increase in both fluorescence intensity and anisotropy as K⁺ enters the membrane, owing to a reduction of collisional quenching of the dye and a decrease in the dynamic motions of the dye upon entry into the bilayer lipid membrane.^{72,73}

Initial attempts to monitor ion channel activity using Safranine O in the external solution were successful for reconstituted gA in solution. However, significant problems arose when the assay was attempted for reconstituted gA that was entrapped in DGS-derived glasses. For example, the dye was observed to have irreproducible responses from sample-to-sample, likely owing to direct interactions of the cationic dye with the anionic surface of the silica which precluded association of the probe with the membrane.⁷⁴ Furthermore, the addition of KCl to the sol often led to leaching of some of the dye, further interfering with the response of the dye to membrane potential and leading to the need to include the probe within the KCl solution to avoid dilution of the probe.

To overcome these problems, it was necessary to locate the Safranine O within the interior of the liposome only, as shown in FIG. 2b. In this case, the influx of potassium ions again results in a positive interior and a negative exterior for the liposomes. However, the Safranine O will now respond by partitioning from the membrane into solution owing to repulsion by the net-positive charge, leading to a decrease in both fluorescence intensity and anisotropy upon formation of an ion gradient. This assay format avoids association of the dye with the silica, and has the added advantage of allowing the liposomes to be formed with no internal salt so as to maximize the ion gradient that can be generated upon addition of a salt solution. A further alteration of the assay was to use potassium iodide in place of potassium chloride to generate the ion gradients. Iodide is a well-known quencher that is membrane impermeable, thus iodide abolishes any contribution to the fluorescence intensity from residual Safranine O that is on the exterior of the liposome, enhancing the overall response from the probe that is located inside the membrane.

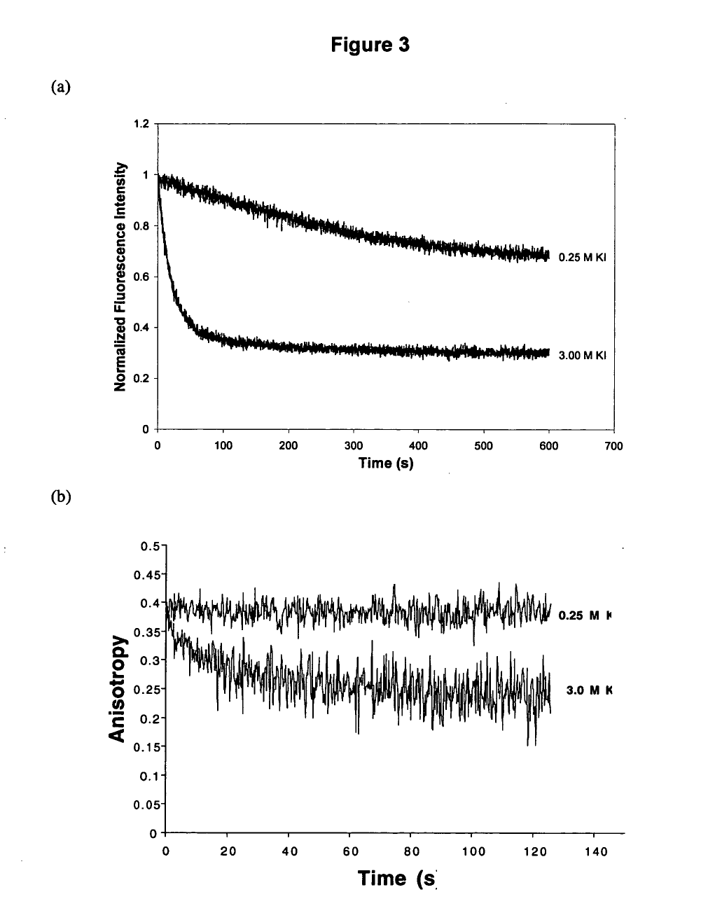

FIG. 3 shows the changes in both fluorescence intensity (Panel A) and anisotropy (Panel B) that were obtained for reconstituted gA within DGS derived silicate upon addition of low and high levels of KI. Both the intensity and anisotropy decrease upon addition of KI, with the magnitude of the decrease becoming larger at the higher level of KI, as expected. These responses are consistent with the repulsion of the dye from the hydrophobic membrane owing to the influx of K⁺ into the membrane, and provide evidence that ion channel activity can be monitored for reconstituted ion channels even after entrapment into sol-gel derived silica, proving that both the membrane and the ion channel are able to withstand the entrapment conditions.

To examine the effects of immobilization on the ion channel activity of gramicidin A, ion flux was monitored for reconstituted gA both in solution and after entrapment to allow a direct comparison of the fluorescence responses. For assays performed in solution, liposomes that contained gA and an internal solution of safranine O were added to solutions of KI, and the changes in emission intensity were immediately measured. This method avoided dilution of the sample, as would occur if KI were added to a liposome solution, making it possible to accurately determine the initial intensity of the solution before the ion flux began. In the case of entrapped gA, the KI was added to the top of the monolith within the microwell plate to initiate a response. In this case, the liposomes were not diluted and thus the determination of the initial intensity was straightforward.

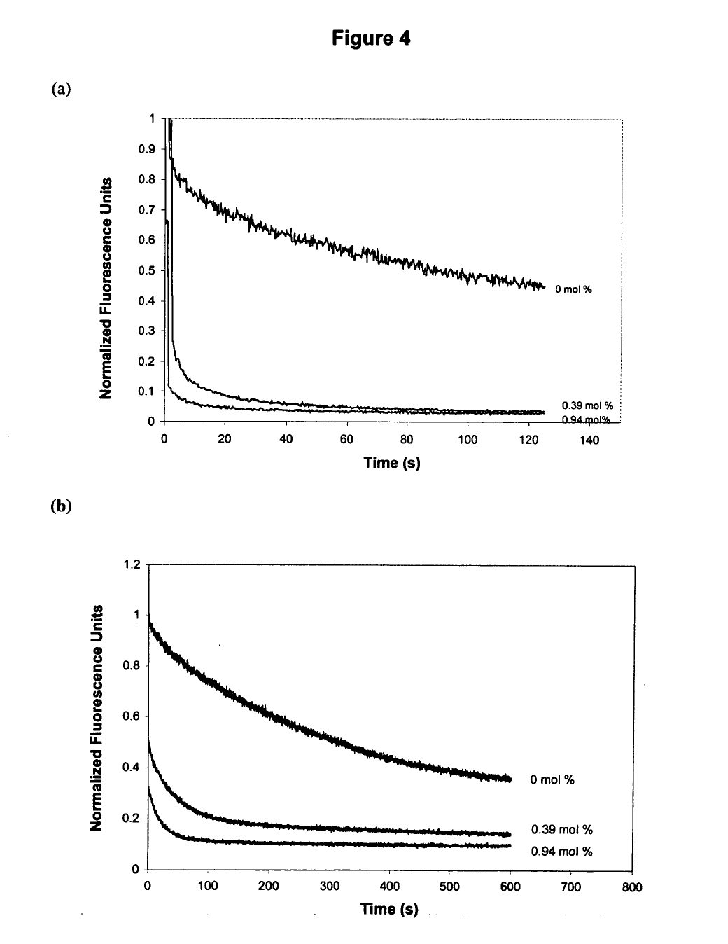

FIG. 4 shows the response of Safranine O to development of membrane potential for liposomes that contained varying levels of gA, both in solution and following entrapment. Even in the absence of gA, there is a significant fluorescence response that is due to the passive transport of K⁺ directly through the lipid membrane. However, it is apparent that incorporation of gramicidin A into the phospholipid membrane results in development of a much larger potential at much faster rates over the time-course of the experiment, and that the response is increased in rate and magnitude as the level of gA increases. The results clearly show that entrapped gA exhibited a similar response to the development of membrane potential as those in solution, except that the rate and the final magnitude of the response were lower for the entrapped sample, likely owing to diffusional limitations for transport of K⁺ into the membrane, and a lower overall level of free K⁺ within the glass owing to electrostatic interactions with the anionic surface of the silica.⁷⁵

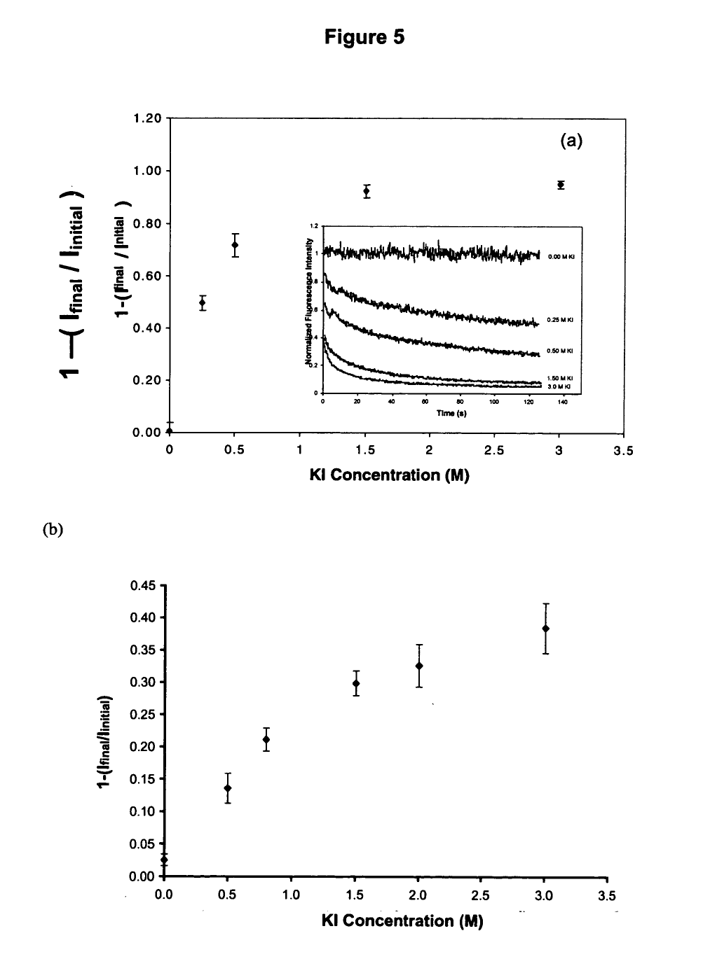

After establishing that incorporation of reconstituted gramicidin A into the membrane resulted in a viable system for the generation of transmembrane ion fluxes, further investigations were carried out to examine the effects of different potassium ion concentrations on the development of the membrane potential. FIG. 5 shows the response of DOPC liposomes containing 0.93 mol % gramicidin to a range of K⁺ concentrations. As expected, the rate at which the emission intensity changes and the extent of the overall fluorescence response both increased as higher salt concentrations were introduced. Both solution and sol-gel entrapped samples exhibited the same trend of a concentration-dependent increase in response; however, the maximal response from the entrapped samples was again slightly lower than that measured in solution, in agreement with the results presented above.

### EXAMPLE 3

### Inhibitors of gA Ion Channel Activity

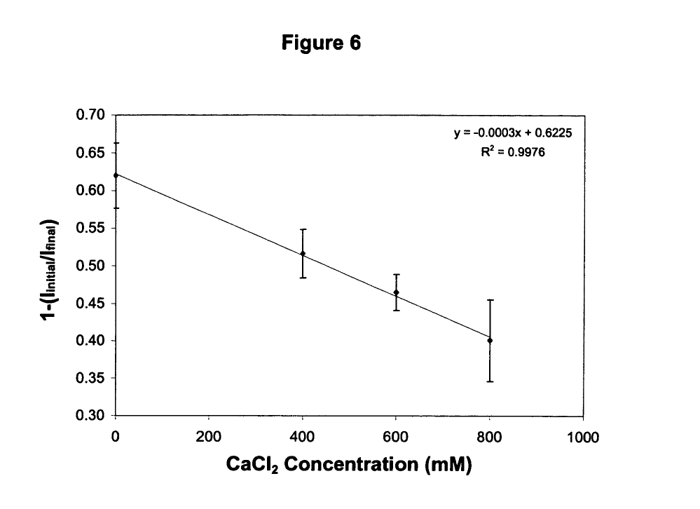

A final test of the potential utility of the entrapped gA ion channel was to assess whether the ion channel activity could be inhibited by addition of channel blocking agents. It has been well established that the presence of divalent cations inhibits the flux of potassium and sodium ions through gramicidin by blocking their passage through the channel.⁷⁶ Inhibition of reconstituted gA entrapped in DGS derived silicate was examined by adding various levels of CaCl₂ to the entrapped samples along with 3.0 M KI. As shown in FIG. 6, the presence of calcium ions produces a significant and concentration-dependent decrease in the potential induced fluorescence response to ion flux, consistent with inhibition of the ion-channel activity. The inhibitory effect requires the presence of several hundred millimolar of Ca²⁺, which in expected given that Ca²⁺ must compete with molar levels of K⁺ for access to the ion channel. A benefit of the “inverted” Safranine O assay is that it avoids the potential for the direct interaction of Ca²⁺ with the fluorescent probe. Akerman et al have demonstrated the addition of divalent cations can directly inhibit the ability of safranine to embed into the membrane. However, entrapping the dye within the liposome leads to the exclusion of Ca²⁺ from the vicinity of the probe. Therefore, any change in response of safranine resulting from the presence of divalent cations cannot be a direct effect of the ions alone but must be due to inhibition of ion passage through the gramicidin ion channel. To confirm this assertion, the same assay was performed without incorporation of gramicidin into the membrane. The results confirmed that no decrease in response occurs upon addition of CaCl₂, ruling out direct interactions of Ca²⁺ with safranine O.

### EXAMPLE 4

### Stability of Entrapped gA Ion Channel Protein

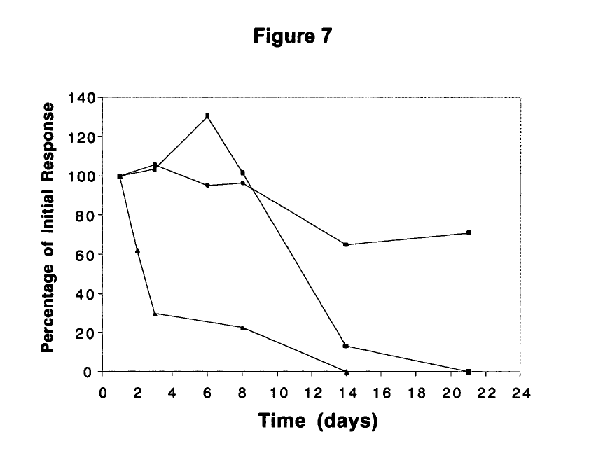

A key advantage of entrapping biomolecules is the potential for improving the long-term stability of the biomolecule.³⁸ To characterize the stability of entrapped gA, several samples were aged at 4° C. over a period of several weeks in air, in the presence of external aqueous buffer, or in the presence of 25% glycerol. FIG. 7 shows the effects of the various aging conditions on the response of safranine to the development of a transmembrane potential. The results show that dry-aged samples lost almost half of their initial activity after two days, and approximately 75% of the initial activity after three days of aging. The instability upon aging in air is an obvious result of loss of water from the system, which leads to dehydration and rupture of the liposomes. On the other hand, samples that were aged either in buffer or in the presence of glycerol maintained their initial activity for over a week, suggesting that the entrapped ion channel may be sufficiently stable to allow the development of protein microarrays or bioaffinity columns that can be used for screening of agonists and antagonists of membrane-bound proteins.

### EXAMPLE 5

### Radioassays of Ligand Binding by Entrapped nAChR

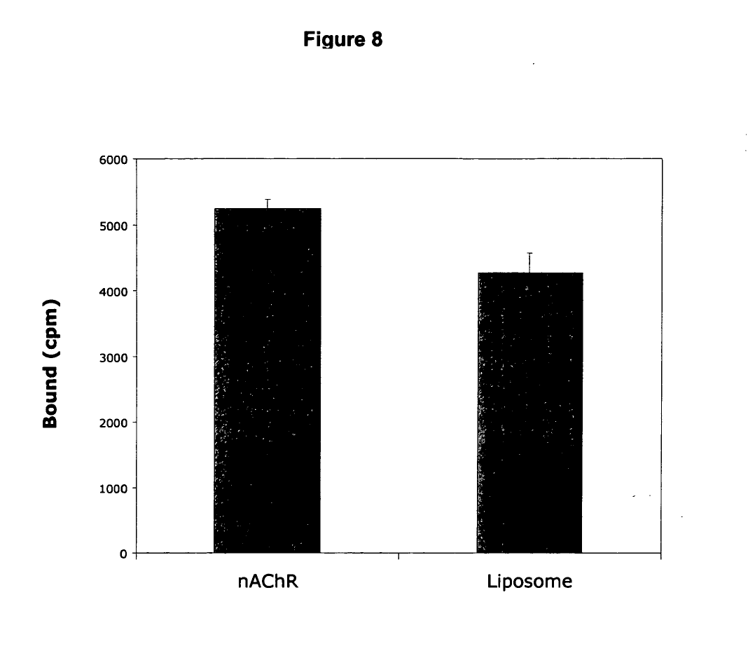

To assess the ligand binding activity of entrapped nAChR we examined both IMR-32 and Torpedo californica nAChR when entrapped in either DGS (IMR-32 nAChR) or sodium silicate (Torpedo californica nAChR) derived silica. FIG. 8 shows the response obtained upon addition of the radioligand ³H-epibetadine to Torpedo californica nAChR entrapped in sodium silicate derived silica (Panel A) and the response obtained for blank liposomes entrapped in sodium silicate derived materials (Panel B). The results clearly show that there is statistically significant specific binding to the receptor, although the non-specific binding of the radioligand to the silica surface remains a problem. The total specific binding of the entrapped receptor (ca. 1000 cps) is approximately 25% the specific binding activity obtained for free AChR (ca. 4000 cps), indicating that a significant fraction of the entrapped nAChR is either denatured or inaccessible to analyte. However, the amount of specific binding is more than sufficient to conclusively prove that a fraction of the receptor remains active after entrapment.

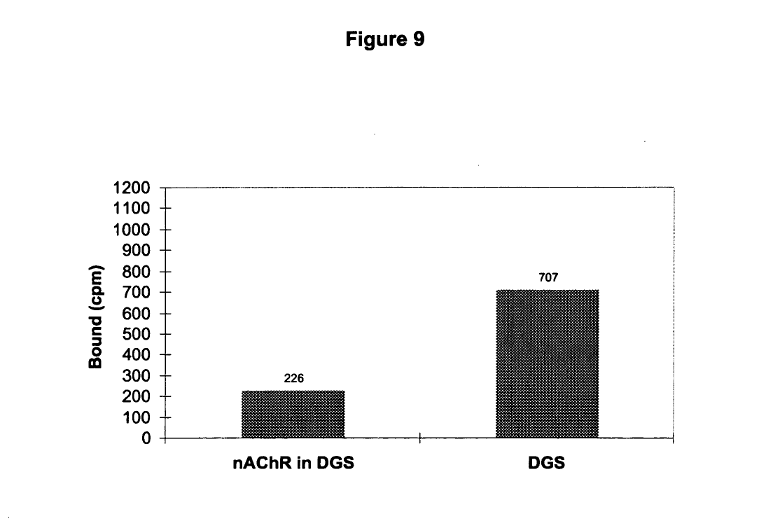

FIG. 9 shows the specific binding of ³H-epibetadine to IMR-32 nAChR when entrapped in DGS derived materials relative to the binding obtained in the absence of entrapped nAChR. In this case the amount of specific binding is ca 500 cps, which is about half the amount observed for nAChR in sodium silicate glasses. No activity was observed from Torpedo californica nAChR in DGS derived glasses, suggesting that sodium silicate based materials may be superior for entrapment of nAChR.

FIG. 10 shows the results of a competitive binding assay wherein varying concentrations of a non-radioactive antagonist (d-tubocurarine, Panel A) or agonist (nicotine, Panel B) were introduced along with a constant concentration of ³H-epibetadine to IMR-32 nAChR entrapped in DGS derived materials. In each case the residual radioactivity resulting from bound radioligand was decreased as the concentration of non-radioactive ligand increased, as expected. More importantly, the IC₅₀ and K_{I} values for both d-tubocurarine and nicotine are in good agreement with those obtained from solution based experiments, and are in relatively good agreement with literature values, showing that the entrapment process does not dramatically alter the dissociation constants for the entrapped nAChR. The key drawback of the radioligand binding assay is that a similar response (i.e., decrease in radioactivity) is observed upon binding of either agonists or antagonists, and thus no discrimination of the functional response of the nAChR to such ligands can be done. To overcome this problem an assay based on enhancement and diminution of ion channelling was developed to provide more detailed information on the mode of action of the ligand, as described below.

### EXAMPLE 6

### Modulation of nAChR Ion Channelling by an Antagonist

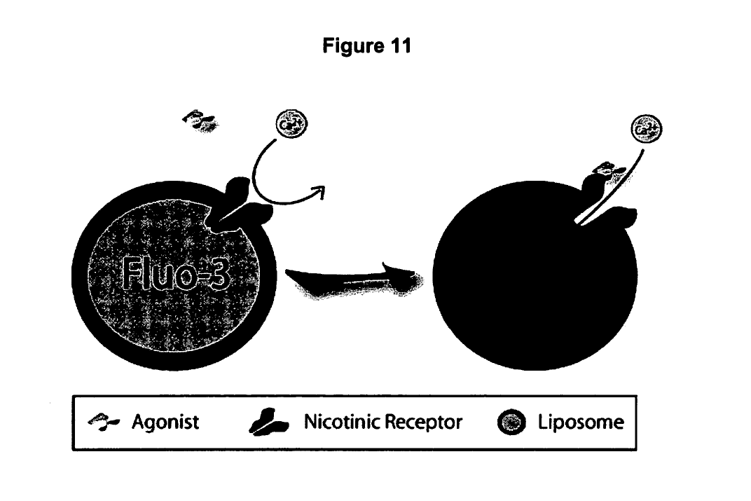

FIG. 11 shows the concept of the fluo-3 based assay for measuring the Ca(II) ion flux across nAChR doped liposomes, which is based on the enhancement in the emission intensity of fluo-3 upon binding of Ca(II). In the absence of an agonist the channel remains closed and no ion flux is observed. Upon binding of an agonist the nAChR ion channel opens and Ca(II) can pass into the membrane, resulting in a large increase in emission intensity from intraliposomal Fluo-3.

FIG. 12 shows the changes in emission intensity of intraliposomal fluo-3 with time (Panel A) and the normalized concentration-dependent decrease in fluo-3 emission intensity (Panel B) due to blockage of the passage of Ca(II) ions upon addition of varying levels of the antagonist d-tubocurarine to n-AChR doped liposomes entrapped in DGS derived glasses that were previously incubated with an excess of the agonist nicotine to cause channel opening. The decease in emission intensity correlates to a decrease in ion flux owing to closing of the nAChR channel upon binding the antagonist. The results show that in the absence of antagonist, the presence of nicotine produced the expected rapid increase in fluorescence intensity upon addition of Ca(II). However, in the presence of the antagonist d-tubocurarine, the response is reduced owing to the blockage of a portion of the ACHR ion channels. The signal eventually reaches the same intensity plateau as is observed for liposomes containing no nAChR, indicative of full blockage of the ion channel. As shown in Panel B, the response is concentration-dependent, and thus will be useful for screening of antagonists against nAChR. This response indicates that the entrapped AChR channel can be modulated by antagonists, showing that the AChR:liposome assembly entrapped in sol-gel glass is suitable for drug-screening studies.

### EXAMPLE 7

### Modulation of Entrapped AChR Ion Gating Using an Agonist

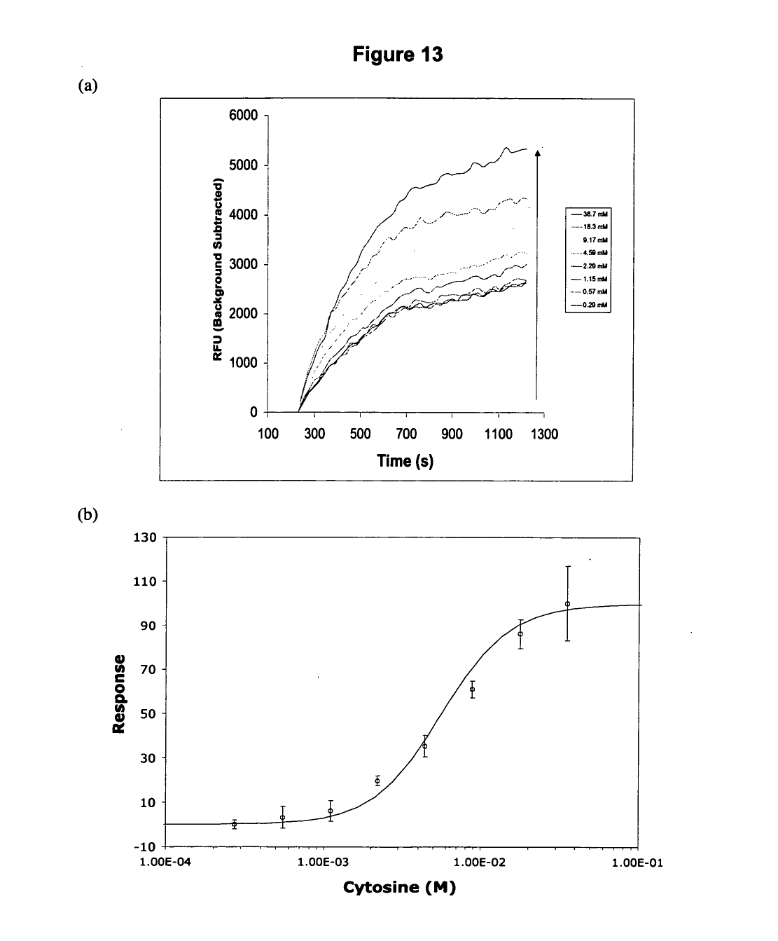

FIG. 13 shows the changes in emission intensity of intraliposomal fluo-3 with time (Panel A) and the normalized concentration-dependent decrease in fluo-3 emission intensity (Panel B) due to enhanced passage of Ca(II) ions upon addition of the agonist cytisine to nAChR doped liposomes entrapped in DGS derived glasses that were previously incubated with an excess of the antagonist d-tubocurarine. The increase in final emission intensity upon addition of Ca(II) in the presence of higher levels of cytisine correlates to an increase in ion flux owing to opening of the nAChR channel upon binding the agonist. Panel B shows that the increase in intensity occurs in a manner that depends on the concentration of cytisine added. The increase in ion flux provides clear evidence that the cytisine acts as an agonist and thus opens the AChR ion channel, producing a Ca(II) flux across the membrane. On the other hand, the decrease in signal upon addition of d-tubocurarine, described in Example 6, provides evidence that this ligand acts as an antagonist. These results prove that the ion-flux assay can discriminate agonists from antagonists and also proves that nAChR remains active upon entrapment and capable of generating transmembrane ion fluxes in an agonist- or antagonist-dependent manner.

### EXAMPLE 8

### Ca(II) Ion Flux Measurements Using Ionomycin:Liposome Assemblies

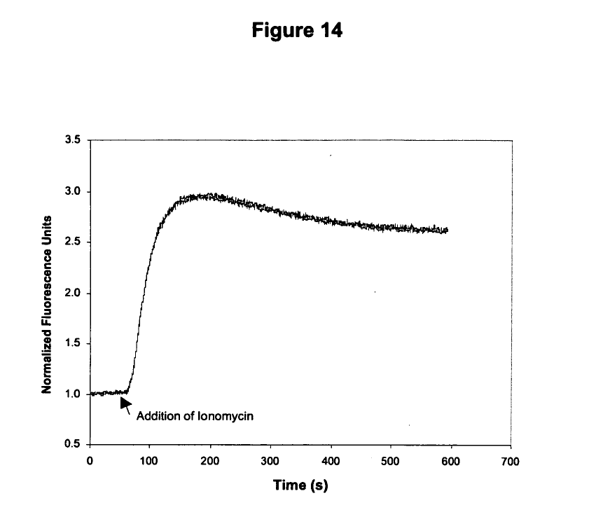

FIG. 14 shows the fluorescence intensity response of the calcium selective indicator dye Fluo-3 to the influx of calcium into DOPC liposomes in buffered solution following the addition of a calcium selective ionophore ionomycin to the membrane. In this case, Ca(II) is initially present only outside the liposome, while fluo-3 is present only inside the liposome. The addition of ionomycin results in the incorporation of the ionophore into the membrane, and produces a channel through which Ca(II) can move into the interior of the liposome. The movement of Ca(II) into the liposome causes the fluo-3 response to increase dramatically (3-fold), producing some hyperpolarizability of the membrane, followed by a slight reduction in intensity as the Ca(II) concentration equilibrates across the membrane. This example clearly shows that the entrapped dye can be used to monitor Ca(II) ion flux.

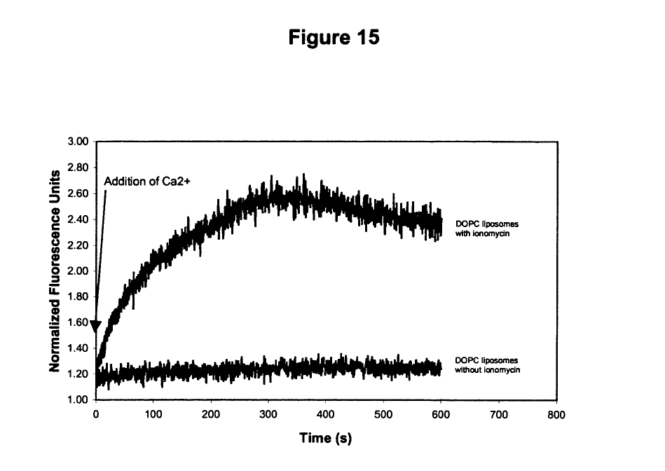

FIG. 15 shows the response of fluo-3 to the addition of calcium ions for DOPC liposomes both with and without ionomycin present within the membrane following entrapment in sodium silicate derived silica. The data clearly show that the presence of ionomycin results in the formation of a pore within the lipid membrane, which in turn produced a flux of Ca(II) from the exterior to the interior of the liposome upon addition of Ca(II) to the entrapped liposome. The data confirm that the ionomycin-liposome-Ca(II) system can be used either for detection of ionomycin or detection of Ca(II) using transmembrane Ca(II) flux, and the resulting fluorescence intensity change, as a signal. The data clearly show that the entrapped liposomes are intact, as there is no response in the absence of ionomycin, and that the ionophore is membrane-associated, producing the desired fluorescence signal when Ca(II) is added.

### EXAMPLE 9

### Liposome Microarrays Using Transmembrane Ion Flux Signalling

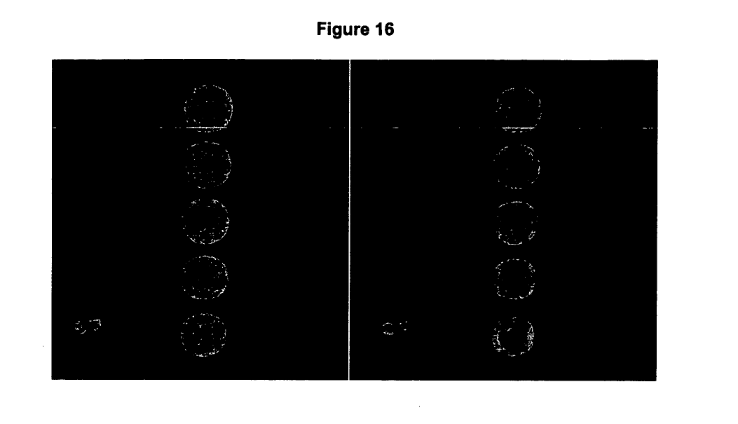

An extension of the Fluo-3 based ion channel activity assay is the microarray format. Pin-printed sol-gel derived microarrays were constructed from samples illustrated in example 5. The microarrays were constructed with both negative and positive controls present. Negative controls consisted of buffered sodium silicate glass or fluo-3 loaded DOPC liposomes without ionomycin, while the positive control was entrapped fluorescein-dextran. In addition, the array contained fluo-3 loaded DOPC liposomes with ionomycin present within the membrane bilayer. It was clearly seen that upon addition of calcium ions to the exterior of the pin-printed array that only the samples containing the ionomycin ion channel underwent a change in fluorescence intensity, consistent with transmembrane ion flux and a corresponding increase in fluo-3 intensity (FIG. 16). This example shows that the microarray format can be used to deposit intact liposome-ionophore assemblies onto surfaces, and to probe a functional response (i.e., transmembrane ion flux). Based on the other examples presented above, it is clear that such a microarray formation and readout method can be directly transferred to ligand gated ion channels such as the nicotinic acetylcholine receptor.

### EXAMPLE 10

### Entrapment of nAChR in Macroporous Silicates

Purification of nAChR from Torpedo californica: Two purifications were performed on separate portions of electroplax tissue from Torpedo californica (Aquatic Research Consultants, San Pedro, Calif.) following procedures described previously by Raftery et al.⁵⁹ The nAChR rich vesicles were then divided into 100-400 μL fractions and frozen and stored at −86° C. until needed.

Determination of Receptor Concentration: Receptor Binding site concentration was determined by ultra-filtration radio-assay performed in the wells of a 96-well, 1.0 μm glass fiber filter plate (Millipore Corp., Mississauga, ON). Receptor stock was first diluted then dispensed into the wells of a filter plate that had been pretreated with 3% polyethylenimine. Total binding was determined by addition of a saturating amount (3.5 nM final concentration) of ³H-epibatidine (Amersham Biosciences Buckinghamshire, UK), non-specific binding was determined at the same concentration of ³H-epibatidine plus 1 mM (−)-nicotine. The free ³H-epibatidine was separated by 5 rapid washes with ice-cold buffer using vacuum filtration. The filters were then individually punched from the filter-plate soaked in scintillated fluid and counted to obtain the specific and non-specific bound counts. The receptor binding sites of the stock obtained from the first purification was 24±5 nM, and the second purification yielded a stock receptor concentration with 60±2 nM binding sites.

Entrapment of nAChR: Diglyceryl silane (DGS) (provided by Dr. Michael Brook of McMaster University and prepared as described elsewhere⁶⁰) derived sol-gels were prepared by adding 1.0 g of solid DGS to 1500 μL of distilled deionized water followed by sonication at 0° C. for 1.0 hour until all of the silane precursor had been dissolved and the solution had become homogeneous and transparent. Samples derived from sodium silicate were prepared as described previously.⁷⁷ Briefly, the sol-precursor was prepared by adding 1.39 g of sodium silicate to 5.0 mL of ddH₂O. This solution is then passed through a strong cation exchange resin (Dowex) to yield a sol solution with a pH of 4.0. Equal volumes of the buffered ACHR solution and the hydrolyzed DGS or sodium silicate solution were mixed and the mixture was immediately placed into 96 well plates in 40 μL aliquots and allowed to gel. Blank samples used for equilibrium dialysis experiments were prepared as described above by replacing the receptor stock with an aqueous solution that contained ca. 40 mg/mL of Asolectin® (Sigma, St. Louis, Mo.) liposomes.

Macroporous materials were prepared as follows: a nAChR stock solution was mixed with an equal volume of buffered solution containing 32% PEO (w/v) and, in some cases, 1.2% APTES (w/v). This buffer/PEO/APTES/protein mixture was then mixed rapidly with an equal volume of hydrolyzed silane (DGS or sodium silicate) and the mixture was immediately placed into 96 well plates in 40 μL aliquots. The final composition was of the solution was 8% w/v PEO (10 kDa), 0.3% w/v APTES and 6-15 nM nAChR binding sites (240-600 fmol of binding sites per monolith). Blanks were prepared as described above, and contained a final concentration of 10 mg/ML Asolectin liposomes. All materials were aged for a minimum of 1 hour at 4° C. before use.

Radioassays of nAChR: Following aging, 10 μL of either buffer or 10 mM nicotine was added to the silica monoliths and allowed to incubate at 4° C. for 2.5 hrs. 80 μL of ³H-epibatidine in buffer was added to the monoliths to a final concentration of 1.0-3.0 nM, and incubated for 18 hr at 4° C. After incubation, 75 μL of ³H-epibatidine solution was drawn off the top of the monolith and dissolved in 20 mL scintillation fluid. The radioactive decay from ³H-epibatidine was then counted to determine the ratio of free ligand existing in solution. Nicotine was added to determine the amount of specific binding to the receptor itself, and the Asolectin liposome samples were used to evaluate the amount of non-specific binding to the matrix.

Determination of Binding Constants: For competitive assays, 10 μL of various concentrations (−)-nicotine or (+/−)-epibatidine were added to the tops of the monoliths containing nAChR or Asolectin liposomes and allowed to incubate for 2.5 hrs. 80 μL of 3.0 nM ³H-epibatidine was then added and the samples were incubated for 16 hrs. Free ligand was determined as described above. Alterations to the general procedure included rapid mixing of buffered samples with hydrolyzed sol in a microtiter plate shaker, which yielded exceptionally homogeneous monoliths sample-to-sample. Also, after incubation samples were subjected 15 min of gentle shaking in an incubator at 37° C. The specific and non-specific binding was then measured as described above. The analogous filter-plate based solution experiments with nicotine and epibatidine were performed in parallel. Data was interpreted using the Hill equation.

Data Analysis: For silica samples data was normalized as follows. Counts drawn from 75 μL of solution from the tops of the monoliths (either nAChR of liposomes) were subtracted from the total counts from ³H-(−)-epibatidine added to the sample, giving the apparent bound counts. The apparent bound counts from the liposomes were then subtracted from the apparent bound counts from the receptor. The residual counts at the highest levels of competitor were then subtracted from all other samples at the various concentrations to bring the base-line counts to zero. The counts are then normalized as a percentage between zero and the maximum counts observed at low competitor concentrations. All samples shown are an average of four independent samples. For solution-based samples the bound counts were determined directly from measurement of the filters used to separate the receptor bound from unbound ligand. The bound counts were then baseline subtracted from the minimum residual counts obtained at highest level of competitive ligand, then normalized as a percentage between zero and the maximum level of bound ligand obtained at low concentrations of competing ligand. All values shown are an average of 3 separate samples.

In all cases the binding isotherms were fit to the Hill equation:

$$B = {B}_{0} ( \frac{{{B}_{max} [ L ]}^{n}}{{IC}_{50} + {[ L ]}^{n}} ) \qquad ( 1 )$$

The inflection point of the binding isotherm, deemed the IC₅₀, is a reflection of the binding strength of the competing ligand and depends on the concentration of the ligand which it competes against (in this case ³H-epibatidine) and the K_{D} of that ligand. From these parameters the equilibrium dissociation constant of the competitive ligand (K_{I}) can be calculated using the following expression:

$${K}_{1} = \frac{{IC}_{50}}{1 + \frac{[ {}^{3} H - epibatidine ]}{{K}_{D}}} \qquad ( 2 )$$

Results and Discussion:

nAChR was entrapped into a series of mesoporous and macroporous silica materials and the activity of both free entrapped receptor was determined using equilibrium dialysis radioligand binding assays. Mesoporous materials were prepared from the protein compatible precursors diglyceryl silane^{[78]} and sodium silicate.^{[79]} However, the counts arising from specific binding of ligand to the receptor (receptor specific counts, RSC) were quite low and substantially less than the counts arising from non-specifically retained ligand obtained from the liposome-doped control samples for both DGS and sodium silicate derived monoliths (see FIGS. 8 and 9 for examples—in sodium silicate nAChR shows 5200 total counts on a background of 4300 non-specific counts, thus there is 900 specific counts vs. 4300 non-specific counts). Alteration of the total silica concentration (by inclusion of Ludox into the sol-gel material), or modification of the silica matrix by inclusion of coupling agents (aminopropyltriethoxysilane or gluconamidyltriethoxysilane) to reduce non-specific binding did not have a dramatic effect on either receptor specific binding or non-specific binding, suggesting that the loss in receptor activity was due to either mass transport problems or perhaps to confinement itself.

One of the key differences between membrane-assoicated molecules, including nAChR, and the soluble proteins previously entrapped into mesoporous silica materials is that these molecules are in liposomes that are quite large relative to the average size of the mesopores. In such a case, it is likely that the matrix may either disrupt the lipid bilayer, reducing receptor activity, or may trap the receptor in inaccessible regions of the matrix, decreasing the extent of ligand binding. For this reason, the question of whether silica gels that contained both mesopores and macropores could provide an environment more amenable to entrapment of the receptor was investigated. It is known that certain polymers, specifically polyethylene oxide (PEO), when added to a hydrolyzed silica precursor, can initiate phase separation through the process of spinodal decomposition, leading to the formation of a material containing both meso and macropores.⁸⁰ Previous results from the present inventors have shown that certain soluble enzymes retain activity within this type of nanocomposite material, as indicated by affinity chromatographic techniques.⁸¹

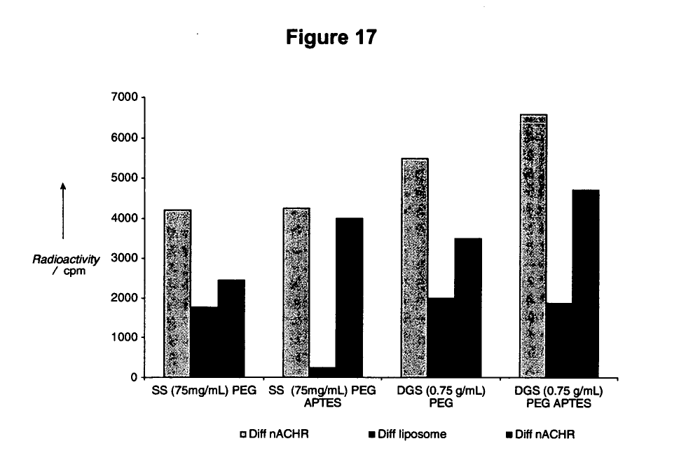

Silica materials were prepared from sodium silicate or DGS containing 8 wt % of 10 kDa PEO to initiate spinodal decomposition and in some cases 0.3 wt % APTES was added to help minimize non-selective retention of analytes.⁸¹,⁸² As shown in FIG. 17, significant receptor-specific binding was observed for all macroporous materials, and in all cases the level of receptor-specific binding was greater that the level of non-specific counts obtained for the liposome-doped materials (negative control samples). While macroporous samples formed from sodium silicate with APTES showed better than 15-fold higher specific binding relative to non-specific binding, it was noticed that both of the APTES-doped samples containing either receptor or liposomes formed heterogeneous gels, which were mechanically unstable. This was not observed for samples that contained only PEO. In addition, the phase separated silica monoliths made from sodium silicate provided much less robust monoliths as compared to the DGS derived materials. It was therefore decided to further examine macroporous DGS-based silica monoliths that were prepared with PEO only.

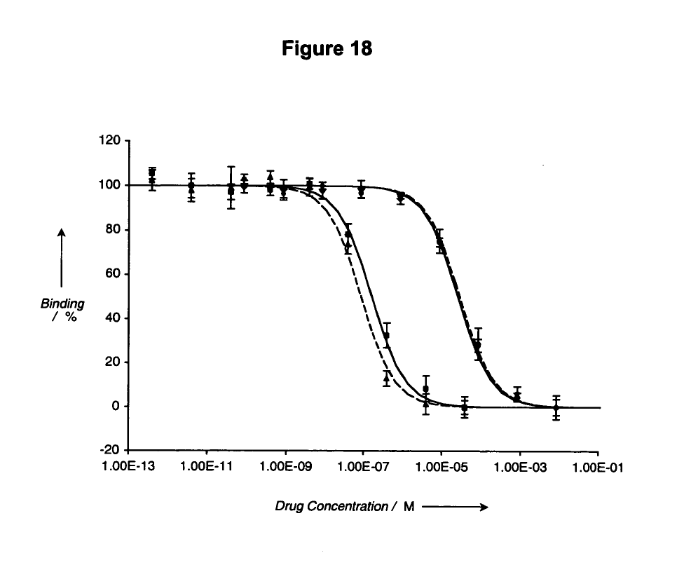

To assess the dissociation constants for the entrapped receptor upon binding to agonists, two drugs were chosen to compete against ³H-epibatidine to obtain full binding isotherms. Non-radioactive (+/−)-epibatidine and (−)-nicotine were used for these studies since both are known agonists of nAChR from Torpedo californica, and they have significantly different K_{d} values such that selectivity could be demonstrated. The binding curves for both free and entrapped nAChR are shown in FIG. 18. The K_{I} values determined for binding of nicotine and epibatidine to entrapped nAChR, were 655 nM and 4.2 nM respectively, while the K_{I} values determined for binding of nicotine and epibatidine to free nAChR under the same conditions were 734 nM and 2.2 nM respectively (Note: typical RSD values for the binding constants are ˜10%). The results show that the binding constants for the entrapped receptor are within error of the solution values. The ligand binding data also provided a means to calculate the total quantity of active receptor, and indicated that approximately 70% of the entrapped receptor remained able to bind to externally added ligand (calculated as 10,000 receptor specific counts for entrapped nAChR vs. 14,000 receptor specific counts for free, solution-based, nAChR when saturated with ³H-epibatidine or nicotine, respectively).

Washing the receptor-containing monoliths repeatedly with buffer and measuring the specific binding of the washes indicated that relatively small amounts (less than 5%) of receptor leached into the aqueous solution surrounding the silica monolith (results not shown). This is likely due to the large size of the liposomes (diameters: 500-1000 nM⁸³) in which the receptor resides relative to the average pore diameter of the meso and macropores in the silica (3.6 nm and 490 nm respectively).

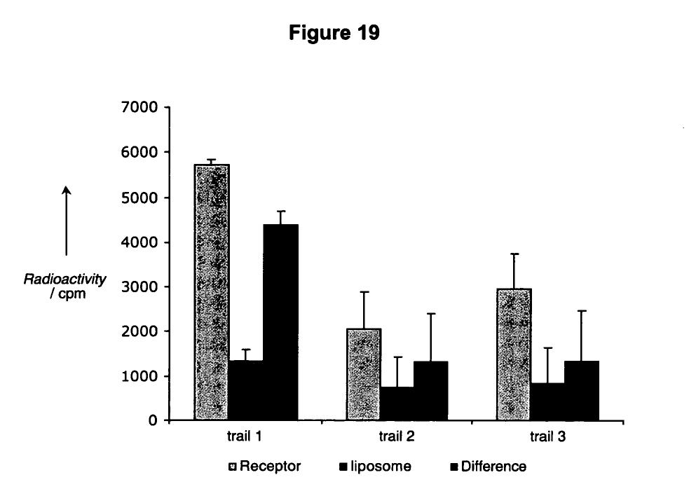

As mentioned previously, the major benefits of immobilization are the ability to improve the storage sability of the receptor and the possibility of reusability, which can reduce consumption of valuable receptor. The entrapped nAChR was observed to remain stable upon long-term storage at 4° C., losing essentially no activity after two weeks of storage (this experiment is ongoing). To determine if entrapped nAChR could be reused, a standard single-point assay was performed three times on the same sample with several vigorous washing steps between trials to remove ligand that was bound to the receptor or the silica matrix. As shown in FIG. 19, significant activity (ca. 70%) was lost after the first assay cycle. However, no further loss in activity was observed in a subsequent assay cycle, although there was a noticeable increase in the error the ligand binding data (n=3). It is not clear whether the decrease in activity is related to the difficulty associated with removal of all radioactive and non-radioactive material between trials, or if the receptor is destabilized throughout the regeneration process.

In summary, the present results demonstrate that entrapment of nAChR into “conventional” mesoporous silica materials derived from sodium silicate or diglycerylsilane leads to minimal activity of the receptor and very high non-specific binding of the ligands to the silica matrix. However, entrapment of nAChR into macroporous silicates formed via spinodal decomposition of added polyethylene oxide (PEO, 10 kDa) leads to high receptor activity (ca. 70% relative to solution) and results in equilibrium disassociation constants (K_{d}) for known agonists of nAChR that are essentially identical to solution values. Moreover, it is evident that samples retain significant activity upon storage and can be reused, and it may very well be possible to fully regenerate samples provided a suitable method can be established.

### EXAMPLE 11

### D2R Binding Assay in Macroporous Silica

D2R Binding Assay in Solution

Human D2S dopamine receptor (D2R) was received from Perkin Elmer Life Sciences (MA, USA) as a membrane suspension in 50 mM Tris-HCl pH.7.4, containing 10% sucrose. The membrane suspension contained 9.9 pmoles of D2R per mg protein, with a total protein concentration is 0.84 mg/ml, corresponding to a receptor concentration of 11.8 nM. Solution-binding assays were carried out using 96 well filtration plates containing 1.0 μm glass fiber, type B filters supplied by Millipore (Multiscreen@ Assay System). The incubation buffer used for the assays consisted of 50 mM Tris-HCl at pH 7.4, containing 120 mM NaCl, 5 mM KCl, 5 mM MgCl₂ and 1 mM EDTA, while the washing buffer contained 50 mM Tris-HCl at pH 7.4 containing 0.9% NaCl. The radioligand, ³H spiperone, was purchased from Amersham Biosciences (Buckinghamshire, UK).

The as received receptor stock solution was diluted by two-fold using incubation buffer, i.e. 45 μl of receptor stock was added to 45 μl of buffer. 20 μl of diluted membrane was used for all ligand binding studies. Prior to each assay, the filter plate was presoaked with 0.5% polyethyleneimine to reduce non-specific binding to the plate. 100 ul of incubation buffer was dispensed into each well followed by either 40 ul of incubation buffer (to assess total radioligand binding) or 40 ul of 1.46 mM haloperidol (0.292 mM final concentration) to test for non-specific binding. A volume of 40 ul of 5 nM ³H spiperone (1 nM final) was then added to all wells followed by addition of 20 μL of the membrane stock to achieve a final receptor concentration of 0.6 nM.

The contents of the wells were incubated for 120 min at 27° C. The contents were then filtered to separate the bound ligands from unbound ligands. Ligands that were loosely adsorbed to the filter paper or to membrane were removed by repeated washing (5×200 μl) with ice cold washing buffer. The combined filtrates were added to 20 ml of scintillation fluid and the radioactivity was counted. Additionally, a control sample consisting of 40 μl of 5 nM ³H spiperone and 1.16 ml of incubation buffer in 20 mL of scintillation fluid was tested to determine the maximum counts that could be obtained in the absence of specific or non-specific binding.

To determine the counts arising from specific binding, the total free counts obtained from samples containing 40 ul of 5 nM ³H spiperone and 0.6 nM D2R is subtracted from the counts obtained from 40 ul of 5 nM ³H Sspiperone with no receptor to obtain the total counts that are associated with binding of ligand to D2R. Following this, the non-specific binding to D2R is determined by first measuring the total free counts from a sample containing 40 μl of 5 nM ³H spiperone, 0.6 nM of D2R and 0.29 mM of haloperidol and subtracting this value from the counts obtained from 40 ul of 5 nM ³H spiperone with no receptor to obtain the total counts that are associated with non-specific binding of ligand to D2R. The receptor specific binding was then determined by subtracting the counts arising from D2R non-specific binding from the total counts arising from binding of the radioligand to D2R.

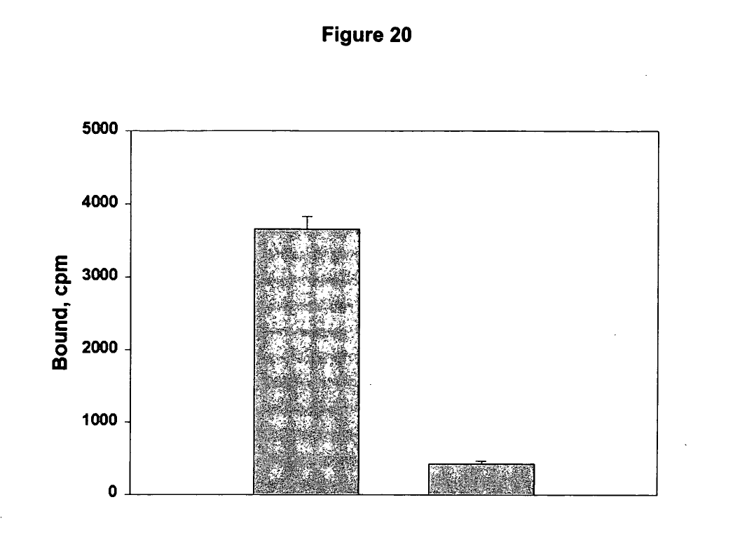

FIG. 20 shows the solution binding assay data. In the PE D2R the total counts (left bar) are 3600 cps, while the non-specific binding is in the range of 500 cps. Thus, the receptor specific binding is about 3000 counts, which is 6-fold higher than the non-specific binding. In addition, the error on the counts is on the order of 10%, which indicates that the D2R assay should be well suited to the determination of ligand binding for entrapped receptor.

D2R Binding Assay in Various Macroporous Silica

Based on the results described above for nAChR, three different silicate compositions were chosen to form DGS-based macroporous silica materials; (i) DGS containing 8% (w/w) PEO (10 kDa), (ii) DGS containing 8% PEO and 1% (w/w) SDS, (iii) DGS containg 8% PEG and 0.3% (w/w) APTES. The macroporous silica samples were prepared using 10 μl of 32% (w/w) PEG or 32% PEG with 4% SDS or 1.2% APTES prepared in 200 mM HEPES buffer, pH 7.4, 10 μl of D2R (not diluted) and 20 μl of a DGS sol that was prepared by adding 1.0 g of DGS to 1.5 ml of water. This led to an amount of receptor that was identical to that used for the solution assays. For blank samples that were used to assess non-specific binding, a 10% sucrose solution (prepared in Tris buffer) was used in place of the D2R sample. In all cases the addition of DGS to the PEG solution resulted in the formation of an opaque solid, indicative of macropore formation. All samples were incubated at 4° C. for two hours before assays were run to allow the silica to set.

Radioassays of entrapped D2R were done by first adding 10 μl of buffer or 0.1 mM haloperidol to the gels (which were present in standard glass filter plates) followed by the addition of 80 μl of 1.625 nM ³H spiperone, to produce a final concentration of 1 nM of radioligand (80 μL is diluted to 130 μL total volume, including the volume of the silica sample) in the sample. The contents mixed briefly and then incubated at 4° C. for 12 hr, followed by further mixing at 37° C. for 15 min to ensure an equilibrium distribution of radioligand was achieved between the solution and solid phases. A 75 μl aliquot was then withdrawn from each sample and 20 ml of scintillation fluid was added, followed by scintillation counting.

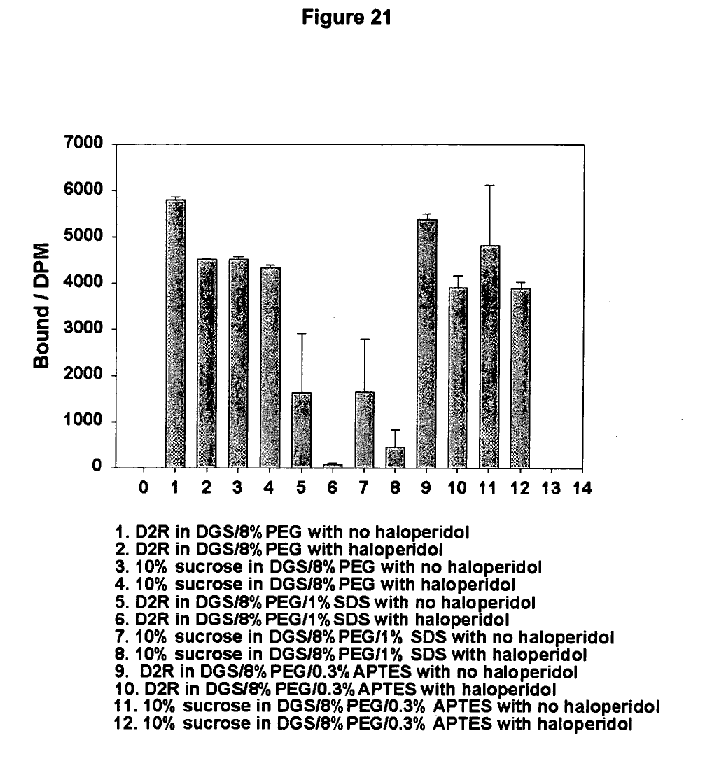

Determination of specific and non-specific binding was done in a manner similar to that described above for the solution assays. First, the total binding to the D2R/silica sample was determined by measuring the radioactivity in the initial 75 μl sample, which was then corrected to the total volume of silica and incubating solutions (130 μl) to account for dilution of the samples. Next, the counts from the supernatant were obtained from a sample that contained D2R that was pre-incubated with haloperidol to displace the specifically bound ligand from the receptor. In addition, counts were obtained from samples that had 10% sucrose in place of D2R which did or did not contain 1 mM haloperidol. This data is shown in FIG. 21 for each of the three sol-gel compositions. The key features that are evident from FIG. 21 are: 1) the significant difference in counts for D2R samples with and without haloperidol in DGS/PEO samples; 2) the relatively high amount of non-specific binding relative to solution (ca 4500 counts in DGS/PEO vs. 500 counts in solution) and; 3) the low level of error in DGS/PEO samples relative to samples containing SDS or APTES. Based on the data in FIG. 21 it is also evident that while additives such as SDS appear to decrease the overall amount of non-specific binding, it also decreases the receptor-specific binding to the point where it appears that there is no activity from entrapped D2R. Addition of APTES has only a minor effect on NSB, and also appears to dramatically reduce receptor activity. Furthermore, the noise level on samples that contain SDS or APTES is much higher than is obtained from DGS/PEO samples, suggesting that these additives may lead to irreproducible formation of silica materials.

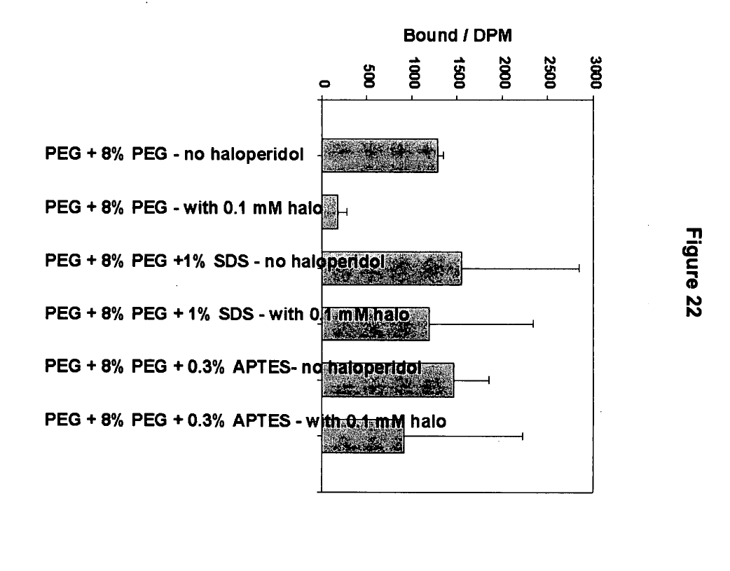

FIG. 22 shows the levels of receptor specific binding and receptor-based non specific binding. The specific D2R binding in macroporous silica is obtained by subtracting the non-specific binding data obtained from macroporous silica containing 10% sucrose solution, i.e., D2R (specific)=(D2R total−10% sucrose-total)−(D2R non-specific−10% sucrose-with haloperidol). Non-specific binding is given by (D2R non-specific−10% sucrose-with haloperidol). The data show that the D2R specific binding in DGS containing 8% PEG is 1100 DPM (total, 1284—receptor-based non-specific, 180). The error bar is relatively low at 176 counts (14%). When considered in light of the total non-specific binding, there is ca. 1300 counts of specific binding on a background of approximately 4500 counts of non-specific binding (FIG. 21). While this is not ideal, the extremely low levels of error in the measurements make it possible to observe a statistically significant level of binding for the entrapped D2R.

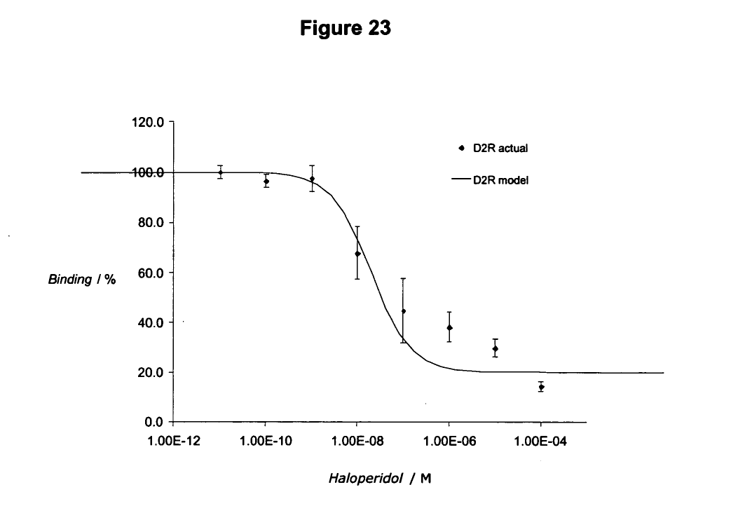

To further assess the properties of the entrapped receptor, a binding isotherm was obtained by measuring the competitive binding of haloperidol against ³H-spiperone to D2R entrapped in DGS containing 8% PEO. FIG. 23 shows the binding isotherm and indicates that the binding of haloperidol to entrapped D2R (indicated by ♦) does lead to displacement of ³H-spiperone in a concentration-dependent fashion. Fitting of the data to the Hill equation (solid line) indicates an IC₅₀ of 20±5 nM for binding of haloperidol to entrapped D2R. Using a binding constant of 130 pM for ³H-spiperone,84 a binding constant (K_{I}) of 2.3±0.5 nM is obtained for haloperidol, which is approximately a factor of 4 higher than the literature value of 0.54 nM. The higher binding constant is likely related to the high amount of non-specific binding of haloperidol to the silica surface.

While the present invention has been described with reference to the above examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

Full Citations for Documents Referred to in the Specification

1. Sreenath Subrahmanyam, Sergey A. Piletsky, and Anthony P. F. Turner;
   Application of Natural Receptors in Sensors and Assays. Analytical. Chemistry. 2002, 74, 3942
2. Tien, H. T.; Ottova, A. L.; From self-assembled bilayer lipid membranes (BLMs) to supported BLMs on metal and gel substrates to practical applications. Colloids and Surfaces, A: Physicochemical and Engineering Aspects 1999, 149,
3. Dong, S.; Chen, X.; Some new aspects in biosensors. Reviews in Molecular Biotechnology 2002, 82, 303.
4. Bernhard, S.; Sleytr, U. B.; S-layer-supported lipid membranes. Reviews in Molecular Biotechnology 2000, 74, 233.
5. W. Knoll, C. W. Frank, C. Heibel, R. Naumann, A. Offenhäusser, J. Rühe, E. K. Schmidt, W. W. Shen and A. Sinner; Functional tethered lipid bilayers. Reviews in Molecular Biotechnology 2000, 74, 137.
6. Krull, U. J.; Brown, R. S.; Hougham, B. D.; McGibbon, G.; Vandenberg, E. T.; Fluorescence wavelength, intensity and lifetime for multidimensional transduction of selective interactions of acetylcholine receptor by lipid membranes. Talanta 1990, 37, 561.
7. Borenstain, V.; Barenholz, Y.; Characterization of liposomes and other lipid assemblies by multiprobe fluorescence polarization. Chemistry and. Physics of Lipids 1993, 64, 117.
8. Watts, A.; NMR of drugs and ligands bound to membrane receptors. Current Opinion in Biotechnology 1999, 10, 48.
9. Sebille, Bernard; Zini, Roland; Madjar, Claire Vidal; Thuaud, Nicole; Tillement, Jean Paul.; Separation procedures used to reveal and follow drug-protein binding. Journal of Chromatography 1990, 531, 51
10. Fang, Y.; Frutos, A. G. Lahiri, J.; Membrane Protein Microarrays. J. Am. Chem. Soc. 2002, 124, 2394
11. Bianco, P.; Protein modified- and membrane electrodes: strategies for the development of biomolecular sensors. Reviews in Molecular Biotechnology 2002, 82, 393
12. Gill, R. T.; Wildt, S.; Yang, Y. T.; Ziesman, S.; Stephanopoulos, G.a Genome-wide screening for trait conferring genes using DNA microarrays. Proc. Natl. Acad. Sci. USA 2002, 99, 7033
13. Su, A. I. Cooke M. P. Ching K. A. Hakak Y. Walker J. R. Wiltshire T. Orth A. P. Vega R. G. Sapinoso L. M. Moqrich A. Patapoutian A. Hampton G. M. Schultz P. G. Hogenesch J. B. Large-scale analysis of the human and mouse
14. Figeys D. Adapting arrays and lab-on-a-chip technology for proteomics. Proteomics 2002, 2, 373
15. Eickhoff H, Konthur Z, Lueking A, Lehrach H, Walter G, Nordhoff E, Nyarsik L, Bussow K. Protein array technology: the tool to bridge genomics and proteomics. Adv Biochem Eng Biotechnol 2002, 77,103
16. Cheng, Y.; Ogier S. D.; Bushby, R. J.; Evans, S. D.; Discrete membrane arrays. Rev. Mol. Biotech. 2000, 74, 159
17. Schriemer D. C.; Bundle, L. L.; Hindsgaul, O.; Micro-Scale Frontal Affinity Chromatography with Mass Spectrometric Detection: A New Method for the Screening of Compound Libraries. Angew Chem. Int. Ed. 1998, 37, 3383
18. Osborn, T. D.; Yager, P. Formation of Planar Solvent-Free Phospholipid Bilayers by Langmuir-Blodgett Transfer of Monolayers to Micromachined Apertures in Silicon. Langmuir 1995, 11, 8.
19. Kallury, K. M. R.; Lee, W. E.; Thompson, M. Enhancement of the thermal and storage stability of urease by covalent attachment to phospholipid-bound silica. Anal. Chem. 1992, 64, 1062
20. Peng, T.; Cheng, Q.; Stevens, R. C.; Amperometric detection of Escherichia coli heat-labile enterotoxin by redox diacetylenic vesicles on a sol-gel thin-film electrode. Anal. Chem. 2000, 72, 1611.
21. Percot, A.; Zhu, X. X.; Lafluer, M.; Immobilization of Lipid Vesicles on Polymer Support via an Amphiphilic Peptidic Anchor: Application to a Membrane Enzyme. Biocong. Chem. 2000, 11, 647.
22. Boukobza, E.; Sonnenfeld, A.; Haran, G.; Immobilization in surface-tethered lipid vesicles as a new tool for single biomolecule spectroscopy. J. Phys. Chem. B. 2001, 105, 12165.
23. Selected references: a) K. L. Martinez, B. H. Meyer, R. Hovius, K. Lundstrom, H. Vogel, Ligand Binding to G Protein-Coupled Receptors in Tethered Cell Membranes. Langmiur 2003, 19, 10925;
24. A. Sévin-Landais, P. Rigler, S. Tzartos, F. Hucho, R. Hovius, H. Vogel, Functional immobilisation of the nicotinic acetylcholine receptor in tethered lipid membranes. Biophys. J. 2000, 85, 141.
25. T. Stora, J. H. Lakey, H. Vogel, Ion-channel gating in transmembrane receptor proteins: functional activity in tethered lipid membranes. Angew. Chem. Int. Ed. 1999, 38, 389.
26. R. Moaddel, L. Lu, M. Baynham, I. W. Wainer, Immobilized receptor- and transporter-based liquid chromatographic phases for on-line pharmacological and biochemical studies: a mini-review J. Chromatogr. B. 2002, 768, 41.
27. D. Stamou, C. Duschl, E. Delamarche, H. Vogel, Self-assembled microarrays of attoliter molecular vessels Angew. Chem. Int. Ed. 2003, 42, 5580.
28. Trojanowicz, M.; Miernik. A. Bilayer lipid membrane glucose biosensors with improved stability and sensitivity. Electrochim. Acta 2001, 46, 1053.
29. Diao, P. et al; Cyclic voltammetry and a.c. impedance studies of Ca2+-induced ion channels on Pt-BLM Bioelectrochem. Bioenergetics 1998, 45, 173.
30. Tien, H. T.; Wang, L.; Wang, X.; Ottova, A. L.; Electronic processes in supported bilayer lipid membranes (s-BLMs) containing a geodesic form of carbon (fullerene C60). Bioelectrochem. Bioenergetics 1997, 42, 161.
31. Asaka, K.; Tien H. T.; Ottova, A. L.; Voltammetric study of charge transfer across supported bilayer lipid membranes (s-BLMs). J. Biochem. Biophys. Methods 1999, 40, 27.
32. Snejdarkova, M.; Rehak, M.; Babincova, M.; Sargent, D. F.; Hianik; Glucose minisensor based on self-assembled biotinylated phospholipid membrane on a solid support and its physical properties. J. Bioelectrochem. Bioenergetics 1997, 39, 235.
33. Gao, Hong; Feng, Jun; An Luo, Guo; Ottova, Angelica L.; Ti Tien, H.; Some Electrochemical Features of Supported Bilayer Lipid Membranes. Electroanalysis, 2001, 13, 49.
34. Yamada, Hiroshi; Shiku, Hitoshi; Matsue, Tomokazu; Uchida, Isamu. Electron transfer at a planar bilayer lipid membrane incorporated with 7,7,8,8-tetracyanoquinodimethane studied by a.c. impedance spectroscopy. 1993, 97, 9546.
35. Lentz, B. R.; Use of fluorescent probes to monitor molecular order and motions within liposome bilayers. Chemistry and Physics of Lipids, 1993, 64, 99.
36. Schuster, B.; Sleytr, U. B. S-layer-supported lipid membranes. Reviews in Molecular Biotechnology 2000, 74, 2.
37. Gill, I. Bio-doped Nanocomposite Polymers: Sol-Gel Bioencapsulates. Chem. Mater. 2001, 13, 3404.
38. Jin, W; Brennan, J. D. Properties and applications of proteins encapsulated within sol-gel derived materials. Anal. Chim. Acta 2002, 461, 1.
39. Sasaki, D. Y.; Shea, L. E.; Sinclair. M. B.; Thin-film silica sol-gels doped with ion-responsive fluorescent lipid bilayers. Proceedings of SPIE-The International Society for Optical Engineering, 1999, 3062, 275.
40. Yamanaka, S. A.; Charych, D. H.; Loy, D. A. and Sasaki, D. Y.; Solid Phase Immobilization of Optically Responsive Liposomes in Sol-Gel Materials for Chemical and Biological Sensing. Langmuir 1997, 13, 5049.
41. Ishiwatari, T.; Shimizu, I.; Mitsuishi, M.; Formation of silicone coated vesicle by sol-gel method. TEM observation. Chemistry Letters, 1996, 33.
42. Nguyen, T.; McNamara, K. P.; Rosenzweig, Z.; Optochemical sensing by immobilizing fluorophore-encapsulating liposomes in sol-gel thin films. Anal. Chim. Acta 1999, 400, 45.
43. Besanger, T. B.; Zhang. Y.; Brennan, J. D. Characterization of Fluorescent Phospholipid Liposomes Entrapped in Sol-Gel Derived Silica. J. Phys. Chem. B, 2002 106(41), 10535-10542
44. Nassif, Nadine; Bouvet, Odile; Rager, Marie Noelle; Roux, Cecile; Coradin, Thibaud; Livage, Jacques. Living bacteria in silica gels. Nature Materials 2002 1, 42
45. Coiffier, Anne; Coradin, Thibaud; Roux, Cecile; Bouvet, Odile M. M.; Livage, Jacques. Sol-gel encapsulation of bacteria: a comparison between alkoxide and aqueous routes. Journal of Materials Chemistry (2001), 11(8), 2039
46. Bergogne, Laurie; Fennouh, Souad; Guyon, Stephanie; Livage, Jacques; Roux, Cecile. Bio-encapsulation within sol-gel glasses. Molecular Crystals and Liquid Crystals Science and Technology, Section A: Molecular Crystals and Liquid Crystals 2000, 354, 79
47. Fennouh, S.; Guyon, S.; Livage, J.; Roux, C. Sol-gel entrapment of Escherichia coli. Journal of Sol-Gel Science and Technology, 2000, 19, 647
48. Fennouh, Souad; Guyon, Stephanie; Jourdat, Catherine; Livage, Jacques; Roux, Cecile. Encapsulation of bacteria in silica gels. Comptes Rendus de l'Academie des Sciences, Serie IIc: Chimie 1999, 2, 625
49 Livage, J.; Roux, C.; Da Costa, J. M.; Desportes, I.; Quinson, J. F. Immunoassays in sol-gel matrixes. Journal of Sol-Gel Science and Technology 1996, 7, 45
50. Weetal, H. H.; Robertson, B.; Cullin, D.; Brown, J.; Walch, M.; Bacteriorhodopsin immobilized in sol-gel glass. Biophys. Biochim. Acta, 1993, 1142, 211
51. Wu, S.; Ellerby L. M.; Cohan, J. S.; Dunn, B.; El-Sayed, M. A.; Valentine, J. S.; Zink, J. I. Bacteriorhodopsin encapsulated in transparent sol-gel glass: a new biomaterial Chem. Mater. 1993, 5, 115
52. Weetal, H. H. Retention of bacteriorhodopsin activity in dried sol-gel glass. Biosensors and Bioelectronics, 1996, 11, 327
53. Sharnansky, L. M., Luong, K. M.; Han, D. Chronister, E. L. Photoinduced kinetics of bacteriorhodopsin in a dried xerogel glass. Biosensors and Bioelectronics, 2002, 17,227
54. Chin, Chen-Ni; Von Heijne, Gunnar; De Gier, Jan-Willem L.; Membrane proteins: shaping up. Trends in Biochemical Sciences 2002, 27, 231
55. Killian, J. A.; Von Heijne, G.; How proteins adapt to a membrane-water interface. Trends in Biochemical Sciences 2000, 259, 429
56. Morillas, Manuel; Swietnicki, Wieslaw; Gambetti, Pierluigi; Surewicz, Witold K.; Membrane environment alters the conformational structure of the recombinant human prion protein. Journal of Biological Chemistry 1999, 274,
57. Muller, Johan M.; van Ginkel, Gijsbert; Van Faassen, Ernst E.; Effect of Lipid Molecular Structure and Gramicidin A on the Core of Lipid Vesicle Bilayers. A Time-Resolved Fluorescence Depolarization Study. Biochemistry 1996, 35, 48
58. Muschol. M.; Dasgupta, B. R.; Salzgerg, B. M.; Caffeine Interaction with Fluorescent Calcium Indicator Dyes. Biophys. J. 1999, 77, 577.
59. Elliot, J.; Blanchard, S. G.; Miller, J. Strader, C. D.; Hartig, P.; Moore, H.; Racs, J.; Raftery, M. A.; Purification of Torpedo Californica Post-Synaptic Membranes and Fractionaltion of their Constituent Proteins. Biochemical Journal. 1980, 185, 667
60. Brook, M. A., Brennan, J. D. Chen, D. Y. U.S. Provisional Patent 60/384,084, 2002.
61. Aurora T S; Li W; Cummins H Z; Haines T H.; Preparation and characterization of monodisperse unilamellar phospholipid vesicles with selected diameters of from 300 to 600 nm. Biochim Biophys Acta 1985, 820, 250
62. Reviakine, I.; Brisson, A.; Formation of Supported Phospholipid Bilayers from Unilamellar Vesicles Investigated by Atomic Force Microscopy. Langmuir 2000, 16, 1806
63. Lasic, Danilo D.; Liposomes in Gene Delivery, 1997 by CRC Press LLC, ISBN:
64. Sodium silicate: Bhatia, R. B.; Brinker, C. J.; Gupta, A. K.; Singh, A. K.; Aqueous Sol-Gel Process for Protein Encapsulation Chemistry of Materials, 2000; 12, 2434-2441.
DGS: Besanger, T. R.; Chen, Y.; Deisingh, A. K.; Hodgson, R.; Jin, W.; Mayer, S.; Brook, M. A.; Brennan, J. D. Screening of Inhibitors using Enzymes Entrapped in Sol-Gel Derived Materials. Analytical Chemistry, 2003, 75, 2382-2391.
65. Rupcich, N.; Goldstein, A.; Brennan, J. D. Optimization of Sol-Gel Formulations and Surface Treatment for the Development of Pin-Printed Protein Microarrays. Chemistry of Materials, 2003, 15, 1803-1811.
66. Brennan, J. D.; Using intrinsic fluorescence to investigate proteins entrapped in sol-gel derived materials. Appl. Spec. 1999, 53, 106A
67. Christiaens B.; Symoens S.; Vanderheyden S.; Engelborghs Y.; Joliot A.; Prochiantz A, Vandekerckhove J.; Rosseneu M.; Vanloo B.; Tryptophan fluorescence study of the interaction of penetratin peptides with model membranes. Eur J Biochem 2002,269,2918
68. Quist, Per-Ola.; 13C solid-state NMR of gramicidin A in a lipid membrane. Biophysical Journal 1998, 75, 2478
69. Woolley, G. A.; Kapral, M. K.; Deber, C. M.; Potential-sensitive membrane association of a fluorescent dye. FEBS Letters, 1987, 224, 337
70. Akerman, K. E.; Saris, N.; Stacking of safranine in liposomes during valinomycin-induced efflux of potassium ions, Biochim Biophys Acta, 1976, 426, 624
71. Bally, M. B.; Hope M. J.; Van Echteld, C. J. A. Cullis, P. R. Uptake of safranine and other lipophilic cations into model membrane systems in response to a membrane potential. Biochim Biophys Acta, 1985, 812, 66
72. Lakowicz J. R.; Principles of Fluorescence Spectroscopy Plenum Press 1983, 233 Spring Str. New York, N.Y. 10013
73. Bhattacharya, Subhash C.; Das, Haritaran; Moulik, Satya P.; Effects of solvent and micellar environment on the spectroscopic behavior of the dye Safranine T. J. Photochem. Photobiol. A, 1994, 84, 39
74. Saleem, M.; Afzal, M.; Hameed, A.; Mahmood, F.; Adsorption studies of cationic dyes on silica gel from aqueous solutions Science International (Lahore) 1993, 5,
75. Flora, K. K.; Brennan, J. D. Fluorometric Detection of Ca2+ Based on an Induced Change in the Conformation of Sol-Gel Entrapped Parvalbumin. Anal. Chem. 1998, 70, 4505.
76. Woolley, G. A.; Wallace B. A.; Model ion channels: gramicidin and alamethicin. J. Membrane Biol. 1992, 129, 10
77. T. R. Besanger, J. D. Brennan, Ion sensing and inhibition studies using the transmembrane ion channel peptide gramicidin A entrapped in sol-gel-derived silica. Anal. Chem. 2003, 75, 1094.
78. Brook, M. A.; Brennan, J. D.; Chen, Y. Polyol-Modified Silanes as Precursorsfor Silica, U.S. Provisional Patent Ser. No. 60/384,084 (to McMaster University), Filed May 31, 2002 and PCT Patent Application S.N. PCT/CA03/00790, Filed Jun. 2, 2003.
79. R. B. Bhatia, C. J. Brinker, A. K. Gupta, A. K. Singh, Aqueous Sol-Gel Process for Protein Encapsulation Chem. Mater. 2000, 12, 2434.
80. H. Minakuchi, K. Nakanishi, N. Soga, N. Ishizuka, N. Tanaka, Octadecylsilylated Porous Silica Rods as Separation Media for Reversed-Phase Liquid Chromatography. Anal. Chem. 1996, 68, 3498.
81. Zheng Zhang Yang Chen, John D. Brennan and Michael A. Brook Methods and Compounds for Controlling the Morphology and Shrinkage of Silica Derived From Polyol-Modified Silanes. PCT Patent Application WO 04/018360, published Mar. 4, 2004.
82. R. Hodgson, Y. Chen, Z. Zhang, D. Tleugabulova, H. Long, X. Zhao, M. G. Organ, M. A. Brook, J. D. Brennan, Protein-Doped Monolithic Silica Columns for Capillary Liquid Chromatography Prepared by the Sol-Gel Method: Applications to Frontal Affinity Chromatography. Anal. Chem. 2004, (in press).
83. Ion Channel Reconstitution C. Miller, Plenum Press; 1986 p 157-273.
84. George, S. R.; Watanabe, M.; Seeman, P. Dopamine D2 receptors in the anterior pituitary: a single population without reciprocal antagonist/agonist states. J. Neurochem., 1985, 44, 1168.
85. PerkinElmer data sheet for human D2Rshort can be found on the internet at: http://las.perkinelmer.com/content/TechnicalInfo/RBHD2CM.pdf

1. A method of immobilizing membrane-associated molecules in silica matrixes comprising combining a liposome-assembly comprising the membrane-associated molecule with a protein- and membrane-compatible sol-gel precursor under conditions which allow a gel to form.

2. The method according to claim 1, wherein the protein- and membrane-compatible sol-gel precursor is selected from an organic polyol silane and sodium silicate.

3. The method according to claim 2, wherein the organic-polyol silane precursor is derived from sugar alcohols, sugar acids, saccharides, oligosaccharides or polysaccharides.

4. The method according to claim 3, wherein the organic-polyol silane precursor is derived from glycerol, sorbitol, maltose or dextran.

5. The method according to claim 4, wherein the organic-polyol silane precursor is selected from diglycerylsilane (DGS), monosorbitylsilane (MSS), monomaltosylsilane (MMS), dimaltosylsilane (DMS) and a dextran-based silane (DS).

6. The method according to claim 5, wherein the organic-polyol silane precursor is diglycerylsilane (DGS).

7. The method according to claim 1, wherein the membrane-associated molecule is selected from non-natural ionophores, ion channel proteins, ion-channel receptors, G-protein coupled receptors, membrane transport proteins or membrane associated enzymes.

8. The method according to claim 6, wherein the membrane-associated molecule is selected from gramicidin, bacteriorhodopsin, the acetylcholine receptor and ionomycin.

9. The method according to claim 1, wherein the liposome comprises phospholipids.

10. The method according to claim 9, wherein the lipid comprises 1,2-dioleoyl-sn-glycero-3-phosphocholine (DOPC).

11. The method according to claim 1, comprising the steps of:
(i) combining an aqueous solution of the protein and membrane-compatible, sol gel precursor with an aqueous solution of a liposome assembly comprising the membrane-associated molecule;
(ii) adjusting the pH of the combination of (i) so that it is in the range of about 4-11.5;
(iii) shaping the combination into a desired shape;
(iv) allowing the combination to gel; and
(v) aging and partially drying the gel.

12. The method according to claim 11, wherein the gel is dried in an aqueous buffer, optionally comprising an effective amount of a humectant.

13. The method according to claim 11, wherein the aqueous buffer comprises about 5% to about 50%% (v/v) of glycerol.

14. The method according to claim 1, wherein the liposome-assembly comprising the membrane-associated molecule and the protein and membrane-compatible, sol-gel precursor are combined in the presence of an indicator molecule and/or in the presence of one or more ligands for the membrane-associated molecule.

15. The method according to claim 1, further comprising combining the liposome assembly and sol-gel precursor in the presence one or more additives which causes spinodal decomposition (phase transition) before gelation.

16. The method according to claim 15, wherein the one or more additives is selected from one or more of water-soluble polymers and one or more compounds of Formula I: 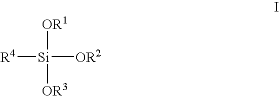
wherein wherein R, R¹ and R³ are the same or different and represent a group that may be hydrolyzed under normal sol-gel conditions to provide Si—OH groups; and R⁴ is group selected from polymer-(linker)ₙ-and 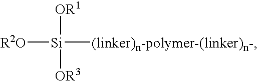
where n is 0 or 1,

17. The method according to claim 16, wherein the one or more additives are selected from one of more water soluble polymers.

18. The method according to claim 17, wherein, the one or more water soluble polymers are selected from one or more of polyethylene oxide (PEO); polyethylene glycol (PEG); amino-terminated polyethylene glycol (PEG-NH₂); amino-terminated polyethylene oxide (PEO-NH₂); polypropylene glycol (PPG); polypropylene oxide (PPO); polyalcohols; polysaccharides; poly(vinyl pyridine); polyacids; polyacrylamides; and polyallylamine (PAM).

19. The method according to claim 18, wherein the one or more water soluble polymers are selected from one or more of PEO, PEO-NH₂, PEG, PPG-NH₂, polyNIPAM and PAM.

20. The method according to claim 19, wherein the one or more water soluble polymers are selected from one or more of PEO, PEO-NH₂ and polyNIPAM.

21. The method according to claim 20, wherein the water soluble polymer is PEO.

22. The method according to claim 21, wherein the PEO has a molecular weight between about 2000-100000 Da.

23. The method according to claim 22, wherein the PEO has a molecular weight of about 10000 Da.

24. The method according to claim 16, wherein the one or more additives are one or more compounds of Formula I.

25. The method according to claim 24, wherein the compounds of Formula I are selected from one or more of compounds of Formula 5: 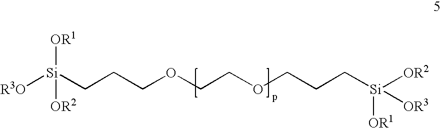
wherein p is an integer between about 4 and 227 and R¹-R³ are the same or different and are selected from C₁₋₄alkyl.

26. A protein- and membrane-compatible sol-gel with a liposome-assembly immobilized therein prepared using the method according to claim 1.

27. A protein- and membrane-compatible sol-gel with a liposome-assembly immobilized therein prepared using the method according to claim 16.

28. A method for the detection of modulators of a membrane-associated molecule comprising:
(a) exposing the protein- and membrane-compatible sol-gel according to claim 26, to one or more test substances; and
(b) detecting a change in one or more characteristics of the membrane-associated molecule,
wherein a change in the one or more characteristics of the membrane-associated molecule in the presence of the one or more test substances compared to a control indicates that the one or more test substances are modulators of the membrane-associated molecule.

29. A method for the detection of modulators of a membrane-associated molecule comprising:
(a) exposing the protein- and membrane-compatible sol-gel according to claim 27, to one or more test substances; and
(b) detecting a change in one or more characteristics of the membrane-associated molecule,
wherein a change in the one or more characteristics of the membrane-associated molecule in the presence of the one or more test substances compared to a control indicates that the one or more test substances are modulators of the membrane-associated molecule.

30. The method according to claim 28, wherein the membrane-associated molecule is an ion channel molecule and the characteristic that is detected is ion flux through the molecule.

31. The method according to claim 29, wherein the membrane-associated molecule is an ion channel molecule and the characteristic that is detected is ion flux through the molecule.

32. The method according to claim 28, wherein the membrane associate molecule is a membrane receptor and the characteristic that is detected is binding of a ligand to the receptor.

33. The method according to claim 29, wherein the membrane associate molecule is a membrane receptor and the characteristic that is detected is binding of a ligand to the receptor.

34. The method according to claim 32, wherein the ligand is radiolabelled.

35. The method according to claim 33, wherein the ligand is radiolabelled.

36. The method according to claim 28, further comprising combining the liposome-assembly comprising the membrane-associated molecule and the protein and membrane-compatible, sol-gel precursor in the presence of an indicator molecule and/or in the presence of one or more ligands for the membrane-associated molecule.

37. The method according to claim 29, further comprising combining the liposome-assembly comprising the membrane-associated molecule and the protein and membrane-compatible, sol-gel precursor in the presence of an indicator molecule and/or in the presence of one or more ligands for the membrane-associated molecule.

38. An improved method for the detection of membrane potentials in a sol-gel entrapped liposome assembly comprising a membrane associated molecule, wherein the membrane-associated molecule is an ion-channel molecule, comprising:
(a) obtaining a solution of the liposome assembly having an indicator molecule located on the interior of the assembly;
(b) removing the indicator molecule from solution external to the liposome assembly;
(c) combining the liposome assembly solution with a silica precursor solution under conditions which allow a gel to form;
(d) contacting the gel with the ion and optionally a test substance; and
(e) detecting a change in the indicator molecule upon transmembrane flux.

39. The method according to claim 38, wherein the indicator molecule interacts with the surface of the sol-gel.

40. The method according to claim 39, wherein the indicator molecule is safranine O.

41. The method according to claim 38, wherein the indicator molecule acts by detecting the ion directly upon entry into the interior of an entrapped liposome.

42. The method according to claim 41, wherein the indicator molecule is a Ca(II) dependent fluorophore.

43. The method according to claim 42, wherein the indicator molecule is fluo-3.

44. The method according to claim 43, where the response of fluo-3 is modulated by agonist or antagonist binding to a ligand-controlled ion gated (LCIG) receptor embedded in the lipid membrane.

45. The method according to claim 44, where the LCIG receptor is nicotine acetylcholine receptor (nAChR).

46. A kit, biosensor, microarray, chromatographic or bioaffinity column comprising the protein- and membrane-compatible sol-gel with a liposome-assembly immobilized therein according to claim 26.

47. A kit, biosensor, microarray, chromatographic or bioaffinity column comprising the protein- and membrane-compatible sol-gel with a liposome-assembly immobilized therein according to claim 27.

48. A method of conducting a target discovery business comprising:
(a) providing one or more assay systems for identifying test substances by their ability to modulate one or more membrane-associated molecules based systems, said assay systems using a method according to claim 28;
(b) (optionally) conducting therapeutic profiling of the test substances identified in step (a) for efficacy and toxicity in animals; and
(c) licensing, to a third party, the rights for further drug development and/or sales or test substances identified in step (a), or analogs thereof.

49. A method of conducting a target discovery business comprising:
(a) providing one or more assay systems for identifying test substances by their ability to modulate one or more membrane-associated molecules based systems, said assay systems using a method according to claim 29;
(b) (optionally) conducting therapeutic profiling of the test substances identified in step (a) for efficacy and toxicity in animals; and
(c) licensing, to a third party, the rights for further drug development and/or sales or test substances identified in step (a), or analogs thereof.
